(19) United States
(12) Patent Application Publication
(10) Pub. No.: US 2026/0151536 A1
(43) Pub. Date: Jun. 4, 2026

(54) SUTURELESS, SELF-RETAINING BIO-TISSUE INSERT FOR THE EYE AND METHODS OF USE

(71) Applicant: Interstat, LLC, White Plains, NY (US)

(72) Inventors: Ravinder D. Pamnani, White Plains, NY (US); Phillip Ianchulev, White Plains, NY (US); Tsontcho Ianchulev, White Plains, NY (US)

(21) Appl. No.: 19/409,442

(22) Filed: Dec. 4, 2025

Related U.S. Application Data
(60) Provisional application No. 63/728,029, filed on Dec. 4, 2024.

(51) Int. Cl. A61L 27/36; A61F 9/00; A61F 9/007

(52) U.S. Cl. CPC A61L 27/3604; A61F 9/0017; A61F 9/00772; A61L 27/3695; A61F 2230/0019; A61F 2230/0023; A61F 2230/0069; A61L 2430/16

(57) ABSTRACT

An amniotic membrane insert configured for self-retaining, sutureless, clear-cornea application to an ocular surface. The insert has an outer edge and an inner edge inside the outer edge. The inner edge defines an opening sized to extend around a circumference of a cornea. The outer edge of the insert has a shape that is non-circular in which a height of the insert is greater than a width of the insert. Related devices, systems, and methods are provided.

### CROSS-REFERENCE TO RELATED APPLICATIONS

This application claims the benefit of priority under 35 U.S.C. § 119(e) to U.S. Provisional Patent Application Ser. No. 63/728,029, filed Dec. 4, 2024. The disclosure of the patent application is incorporated by reference herein in its entirety.

### FIELD

The present technology relates generally to implants and methods for treating an eye, and more particularly, to sutureless, self-retaining bio-tissue inserts for the eye and methods of use.

### BACKGROUND

The chorioamniotic membrane separates the fetus from the mother's endometrium in mammals and includes the amniotic membrane, or amnion, and the chorionic membrane, or chorion. The amniotic membrane is the innermost layer of the chorioamniotic membrane. The amniotic membrane is a thin, transparent tissue that also has several layers, including epithelial layer, a basement membrane, a compact layer, and a fibroblast layer. The amniotic membrane is rich in collagen and various bioactive factors having properties that contribute to the healing process.

The amniotic membrane is obtained from the placenta after childbirth and is known for its use in the treatment of burns and wounds. Amniotic membrane has been used on the ocular surface for its healing and regenerative effects. Key amniotic factors such as Prostaglandin E2 (PGE2), Growth Differentiation Factor 11 (GDF11), Thrombospondin-1, and WNT4 have been identified as major modulators of the healing, anti-inflammatory and immune protective properties of amniotic membrane (see J. Trans. Med. 12, 260 (2014); Cell Res. 24(12):1381-1382 (2014); J. Ocul. Pharmacol. Ther. 31(7):376-385 (2015); Clin. Ophthalmol. 12:677-681 (2018); J. Ophthalmol. 2017; 2017:6404918. doi:10.1155/2017/6404918).

One of the main challenges associated with the clinical use of amniotic tissue on the ocular surface is the difficulty of attaching it non-invasively and durably in a way that does not cause irritation, discomfort and pain in the eye—as it is well established that the ocular/corneal surface is one of the most sensitive tissues in the human body.

Other features and advantages should be apparent from the following description of various implementations, which illustrate, by way of example, the principles of the invention.

### SUMMARY

In an implementation, provided is an amniotic membrane insert configured for self-retaining, sutureless, clear-cornea application to an ocular surface, the insert having an outer edge and an inner edge inside the outer edge. The inner edge defines an opening sized to extend around a circumference of a cornea. The outer edge has a shape that is non-circular in which a height of the insert is greater than a width of the insert.

The non-circular shape of the outer edge can be lentoid, oval, elliptical, pill shape, discorectangle, obround, stadion, egg, or quatrefoil. The opening can be at least 9 mm in diameter. The outer edge can have a maximum longitudinal dimension that is about 15 mm to about 35 mm. The insert can have a width between the outer edge and the inner edge that is at least 100 microns and no greater than 8 mm, or about 1 mm-5 mm, or about 2 mm-4.5 mm.

The insert can include an upper lobe on an upper end region of the insert and a lower lobe on a lower end region of the insert. The upper lobe and the lower lobe can be positioned opposite one another along a longitudinal dimension of the insert and are configured to be received within a superior subpalpebral space and inferior subpalpebral space, respectively. The upper lobe can have a width along the longitudinal dimension between a first point of the outer edge and a first point of the inner edge across from the point of the outer edge. The lower lobe can have a width along the longitudinal dimension between a second point of the outer edge and a second point of the inner edge. The width of at least one of the upper lobe and the lower lobe can be greater than a width of the insert between the outer edge and the inner edge along a lateral dimension perpendicular to the longitudinal dimension. The width of the upper lobe can be equal to, less than, or greater than the width of the lower lobe. The insert can include one or more surface features on one or both of the upper lobe and the lower lobe. The one or more surface features can be a cut-out. The cut-out can extend through at least one of the upper lobe and the lower lobe. The cut-out can extend through the outer edge, but not the inner edge of the at least one of the upper lobe and the lower lobe. The cut-out optionally does not extend through either the inner edge or the outer edge of the at least one of the upper lobe and the lower lobe. The one or more surface features can be configured to allow a fluid to pass from a posterior side of the insert facing the ocular surface while in use to an anterior side of the insert facing away from the ocular surface.

The amniotic membrane can be a multi-layer construct that includes an amnion layer and a chorion layer. The multi-layer construct can be a dual layer of amnion-chorion, chorion-amnion, amnion-amnion, or chorion-chorion. The multi-layer construct can be a triple layer of amnion-amnion-amnion, chorion-amnion-amnion, amnion-chorion-amnion, amnion-amnion-chorion, chorion-chorion-amnion, amnion-chorion-chorion, chorion-amnion-chorion, or chorion-chorion-chorion. The multi-layer construct can be a quadruple layer of amnion-chorion-chorion-amnion or chorion-amnion-amnion-chorion. The multi-layer construct can be dehydrated and rehydrates after positioning against the eye. The multi-layer construct in a dehydrated state can have a first thickness between a posterior surface of the insert and an anterior surface of the insert. The multi-layer construct in a rehydrated state can have a second thickness between the posterior surface of the insert and the anterior surface of the insert that is greater than the first thickness.

The insert can include a retention feature separate from the insert that is configured to provide structural stability to the insert. The retention feature can be formed of a biodegradable or non-biodegradable material. The retention feature can be a bandage or standard corneal contact lens. The retention feature can have an outer perimeter sized to be received within the opening of the insert. The inner edge of the insert can have a diameter that is 0.1 mm-2.0 mm greater than a diameter of the outer perimeter of the retention feature. The insert can have a width between the outer edge and the inner edge. The thickness can be conforming to the retention feature. T the width of the insert can be less than 4 mm. The width of the insert can be less than a thickness of the retention feature.

In an interrelated implementation, provided is an insert made of amniotic membrane that is configured for self-retaining, sutureless, clear-cornea application to an ocular surface. The insert has an outer edge, an inner edge, a first free end, and a second free end opposite the first free end. A length of the insert between the first free end and the second free end and a width of the insert between the outer edge and the inner edge is sized to be received within the cul-de-sac of an eyelid. Optionally, the eyelid can be an upper eyelid or a lower eyelid. The length of the insert can be no greater than 33 mm, the width of the insert can be no greater than 100 mm, and a thickness of the amniotic membrane can be no greater than 5 mm. The insert can further include a retention feature separate from the insert and configured to provide structural stability to the insert. The retention feature can be formed of a biodegradable or non-biodegradable material.

In an interrelated implementation, provided is a method of administering biological factors to an eye including applying amniotic membrane shaped to have an outer edge and an inner edge inside the outer edge, the inner edge defining an opening. The method includes positioning the amniotic membrane onto an anterior surface of the eye so that the opening surrounds a circumference of a cornea of the eye and the inner edge does not contact the cornea; and releasing biological factors from the amniotic membrane to the eye.

In an interrelated implementation, provided is a method of minimally-modifying amniotic membrane tissue into an annular shape configured to be received on an anterior surface of the eye that avoids contacting the cornea. The method includes cutting amniotic membrane tissue into an elongate strip, the strip having a length between a first free end and a second free end, a width across the length, and a thickness; and connecting the first free end and the second free end to form a closed loop, the closed loop defining a central opening sized to surround a circumference of the cornea.

In an interrelated implementation, provided is a method of minimally-modifying amniotic membrane tissue for treatment of an eye including cutting the amniotic membrane tissue into an annulus, the annulus having an outer edge, an inner edge inside the outer edge that defines a central opening sized to surround a circumference of the cornea without contacting the cornea.

In an interrelated implementation, provided is an insert of amniotic membrane coupled to a punctal plug, the punctal plug sized and shaped to insert at least in part within a punctum of an eye to position the amniotic membrane in contact with a surface of the eye.

Optionally, the amniotic membrane is shaped into an elongate strip or disk. The punctal plug can be solid so as to occlude the punctum and reduce drainage of fluid out of the eye. Upon positioning the insert in the eye, the amniotic membrane can be positioned under a lid of the eye. The amniotic membrane is optionally positionable within the cul-de-sac of the eye. The punctal plug optionally includes a proximal collar, a distal tapered region, and a body extending between the collar and the tapered region. The amniotic membrane can be coupled to the collar of the punctal plug by a tissue connector. The insert can optionally include a second punctal plug coupled to the amniotic membrane. The punctal plug and/or the tissue connector can be amniotic membrane.

In an interrelated implementation, provided is an insert of amniotic membrane coupled on a first end to a first punctal plug and coupled on a second end to a second punctal plug. The first punctal plug is sized and shaped to insert at least in part within a first punctum of an eye and wherein the second punctal plug is sized and shaped to insert at least in part within a second punctum of the eye to position the amniotic membrane spanning between the first and the second punctal plugs in contact with a medial anterior surface of the eye. The amniotic membrane is optionally shaped into an elongate strip or disk. At least one of the first and second punctal plugs is optionally solid to occlude the punctum and reduce drainage of fluid out of the eye. The first and second punctal plugs can be amniotic membrane.

In an interrelated implementation, provided is an insert of amniotic membrane in the shape of a punctal plug or wedge. The insert sized and shaped to enter, at least in part, a punctum of an eye. The amniotic membrane is in a dehydrated state and transitions to a rehydrated state after positioning within the punctum. The amniotic membrane in the dehydrated state has a first thickness and the amniotic membrane in the rehydrated state has a second thickness that is larger than the first thickness.

In an interrelated implementation, provided is a method of treating ocular surface disease with amniotic membrane wherein the amniotic membrane is not in contact with the cornea through the entire treatment duration.

In an interrelated implementation, provided is a punctal plug formed of a biotissue having a size and a shape to insert, at least in part, within a punctum of an eye. The biotissue can occlude the punctum. The biotissue can increase a tear film of the eye upon being inserted within the punctum. The biotissue is optionally scleral tissue or corneal tissue.

The biotissue can be amniotic membrane tissue. The amniotic membrane tissue can be a multi-layer construct of amnion and chorion. The multi-layer construct of amnion and chorion can be a dual layer, triple layer, or quadruple layer. The multi-layer construct can be a dual layer of amnion-chorion, chorion-amnion, amnion-amnion, or chorion-chorion. The multi-layer construct can be a triple layer of amnion-amnion-amnion, chorion-amnion-amnion, amnion-chorion-amnion, amnion-amnion-chorion, chorion-chorion-amnion, amnion-chorion-chorion, chorion-amnion-chorion, or chorion-chorion-chorion. The multi-layer construct can be a quadruple layer of amnion-chorion-chorion-amnion or chorion-amnion-amnion-chorion. The biotissue can bioelutes one or more healing factors into a tear film of the eye.

The punctal plug optionally has a tapered portion near a distal end, a collar near a proximal end, and a body portion between the distal end and the collar. At least the body portion can be formed of the biotissue. The collar can be formed of the biotissue. The collar can be formed of a material that is not the biotissue. The biotissue can be dehydrated in a first state having the size. The biotissue can rehydrate upon deployment in the punctum and transitions towards a second state that is a larger in at least one dimension. The biotissue can wedge into and occlude the punctum upon transitioning towards the second state. The shape of the biotissue can be triangular with a narrower dimension toward a distal end and a wider dimension toward a proximal end. The shape of the biotissue can be rectangular or cylindrical. The punctal plug is optionally formed of two or more separate bodies of the biotissue.

The biotissue can increase in one or more dimensions upon deployment in the punctum by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, up to about 250%. The biotissue can have a thickness and a width in a dehydrated state. The thickness in the dehydrated state can be about 25 microns to about 1400 microns and the width in the dehydrated state can be about 25 microns to about 700 microns. The biotissue can have a thickness and a width in a rehydrated state. The thickness in the rehydrated state can be about 50 microns to about 1000 microns and the width in the rehydrated state can be about 50 microns to about 1400 microns.

The biotissue is optionally cut, rolled, and/or folded into the size and the shape. Upon insertion of the biotissue into the punctum, at least a proximal end region of the biotissue can remain outside the punctum. The proximal end region can be shaped into a flap, pad, or collar.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow the disclosure are intended to define the scope of the protected subject matter.

### BRIEF DESCRIPTION OF THE DRAWINGS

These and other aspects will now be described in detail with reference to the following drawings. Generally, the figures are not to scale in absolute terms or comparatively but are intended to be illustrative. Also, relative placement of features and elements may be modified for the purpose of illustrative clarity.

FIG. 1 shows an implementation of a bio-tissue insert;

FIG. 2 illustrates the bio-tissue insert of FIG. 1 positioned on an ocular surface;

FIG. 3 illustrates another implementation of a bio-tissue insert positioned on an ocular surface;

FIG. 4A illustrates another implementation of a bio-tissue insert in an initial cut form factor;

FIG. 4B illustrates the bio-tissue insert of FIG. 4A after fusing into a closed loop having a circular shape;

FIG. 5A illustrates another implementation of a bio-tissue insert in an initial cut form factor;

FIG. 5B illustrates the bio-tissue insert of FIG. 5A after fusing into a closed loop having a polygonal shape;

FIG. 5C illustrates another implementation of a bio-tissue insert in an initial cut form factor;

FIGS. 6A-6F illustrate cross-sectional shapes of the bio-tissue insert of FIG. 1 taken along line A-A;

FIGS. 7A-7C illustrate top plan views of interrelated implementations of bio-tissue inserts;

FIGS. 8A-8G illustrate top plan views of interrelated implementations of bio-tissue inserts;

FIG. 9 illustrates a bio-tissue insert coupled to a retention feature;

FIG. 10A illustrates another implementation of a bio-tissue insert coupled to a retention feature for fixing the insert within the eye;

FIG. 10B illustrates an anterior surface of an eye and the lacrimal system;

FIG. 10C is a schematic of the insert and retention feature of the FIG. 10A fixed within the puncta of the eye;

FIG. 11A illustrates another implementation of a bio-tissue insert prior to rehydration;

FIG. 11B illustrates the bio-tissue insert of FIG. 11A after rehydration;

FIG. 12A illustrates another implementation of a bio-tissue insert prior to rehydration;

FIG. 12B illustrates the bio-tissue insert of FIG. 12A after rehydration.

It should be appreciated that the drawings are for example only and are not meant to be to scale. It is to be understood that devices described herein may include features not necessarily depicted in each figure.

### DETAILED DESCRIPTION

Described herein are bio-tissue inserts, formed for example of amniotic membrane allograft or xenograft, for use on the ocular surface to treat inflammatory, neovascular or other ophthalmic diseases and processes of the eye. The bio-tissue inserts are sutureless and self-retaining. Described are methods of treating ocular surface disease with amniotic membrane inserts wherein the amniotic membrane is not in contact with the cornea through the entire treatment duration.

Therapeutic approaches for delivery of amniotic products to the ocular surface are known. Amniotic membrane corneal shield/bandage lens products are commercially available, such as PROKERA (BioTissue, Inc., Miami, FL), AMBIODISK (Corza Medical, Inc., Westwood, MA) and AMNIOEXCEL (Integra Lifesciences, Inc., San Diego, CA). These corneal shields are exclusively applied on the cornea in the central 10-12 mm of the eye. The shields are circular amniotic tissue discs often attached to the corneal surface using additional retention hardware, such as a bandage contact lens designed to improve stability and retention over the cornea. The amniotic membrane corneal shield typically dissolves after 7-10 days application to the eye as its therapeutic diffusible factors aide the healing of the corneal surface.

A key challenge to the current ocular use of amniotic membrane is that the amniotic corneal disc is positioned over the cornea and causes significant intolerance and pain. This is because the cornea is the most sensitive part of the eye, as it contains unmyelinated nerve endings and the density of nerve endings in the corneal epithelium is 400 times that of the epidermis of the skin (Clinical Anatomy and Physiology of the Visual System (Third Edition), 2012). Additionally, the cornea lacks blood vessels due to the need for transparency, meaning oxygen from the ambient air keeps the cornea healthy. A covering for 7-10 days by an amniotic membrane corneal shield is detrimental to oxygen uptake. In addition, the amniotic membrane is cloudy and optically detrimental to visual axis, which leads to reduced vison and blur.

Amniotic tissue-derived extract in the form of eye drops is another therapeutic approach for providing the healing power of amniotic membrane to an eye. The improvements in ocular surface healing and comfort associated with amniotic membrane transplantation have been attributed, in part, to the diffusible anti-inflammatory properties of the tissue, inhibiting the expression of pro-inflammatory mediators and growth factors. This has been demonstrated in studies of the wound healing applications of amniotic tissue-derived extracts to the ocular surface whereby the amnio cytokines and anti-inflammatory and healing factors (more than 120 of them) were delivered as eye drops without the application of amniotic membrane shield or corneal bandage to the ocular surface (Clinical Ophthalmology 2019:13 887-894). Those cytokines, growth factors, and enzyme inhibitors involved in tissue remodeling (e.g., matrix metalloproteinases) showed dramatic improvement in ocular surface healing and dry eye disease scores. A key challenge with amniotic tissue-derived extracts in the form of eye drops is that it requires daily patient adherence to consistently self-dose the therapy. In one study, adherence to the prescribed dosing regimen in dry eye patients was as low as 10% (Uchino M, et al. Adherence to Eye Drops Usage in Dry Eye Patients and Reasons for Non-Compliance: A Web-Based Survey. J. Clin. Med. 2022 January 12; 11(2): 367).

There remains a significant need for a better method and form-factor for amniotic membrane application to treat the ocular surface for the delivery of therapeutic diffusible amniotic factors, which would not interfere with the visual axis, is associated with minimal or no pain and discomfort, will not dislodge and extrude from the eye, and is not dependent on daily patient adherence.

Described herein are amniotic membrane inserts and methods of using inserts that include amniotic bio-tissue, with or without additional non-tissue structural hardware, for application to the ocular surface. The inserts are designed to avoid impacting the visual axis and avoid the central corneal surface for optimal non-invasive retention and reduced pain and discomfort. At the same time, the amniotic tissue remains resident on the ocular surface to dissolve over time and deliver its therapeutic diffusible factors. Described are methods of treating ocular surface disease with amniotic membrane wherein the amniotic membrane is not in contact with the cornea through the entire treatment duration.

The term “biologically-derived material” or “biotissue” includes naturally-occurring biological materials that are suitable for implantation onto the eye. Biologically-derived material or biotissue includes a material that is a natural biostructure having a biological arrangement naturally found within a mammalian subject including organs or parts of organs formed of tissues, and tissues formed of materials grouped together according to structure and function. Biologically-derived material includes tissues such as amniotic membrane, chorioamniotic membrane, amnion, or chorion. Biologically-derived material includes tissues such as corneal, scleral, or cartilaginous tissues as well as acellular biomatrix tissue. Biologically-derived material includes tissue harvested from a donor or the patient, organs, parts of organs, and tissues from a subject including a piece of tissue suitable for transplant including an autograft, allograft, and xenograft material. Biologically-derived material includes naturally-occurring biological material including any material naturally found in the body of a mammal.

Biologically-derived materials can include naturally-occurring biological tissue including any material naturally found in the body of a mammal that is minimally-manipulated or more than minimally-manipulated according to FDA guidance under 21 CFR § 1271.3(f) such that the processing of the biological tissue does not alter the relevant biological characteristics of the tissue (see Regulatory Considerations for Human Cells, Tissues, and Cellular and Tissue-Based Products: Minimal Manipulation and Homologous Use, www.fda.gov/regulatory-information/search-fda-guidance-documents/regulatory-considerations-human-cells-tissues-and-cellular-and-tissue-based-products-minimal).

The biologically-derived material, sometimes referred to herein as bio-tissue or bio-material, which is used to form the insert can vary and can be, for example, chorioamniotic membrane tissue, amniotic membrane tissue, or amnion, and the chorionic membrane, or chorion, and the umbilical cord. Bio-tissue can alternatively or additionally include corneal tissue, scleral tissue, cartilaginous tissue, collagenous tissue, or other firm biologic tissue. Bio-tissue can include tissue from a mammal, including human and non-human animal tissue.

The inserts described herein can be made of 1% to 100% amniotic tissue with the rest consisting of a bio-degradable or non-biodegradable polymer. The amniotic membrane may consist of amnion only (single layer amnion or dual layer amnion), or may consist of both amnion and chorion layers, or the membrane may consist of umbilical cord tissue. In another embodiment, the membrane sheet thickness may consist of stacked layers of amnion, chorion, or umbilical cord tissues. The amnion has a Young's modulus that is stiffer than chorion (e.g., 21 MPa vs. 2.3 MPa) (see Verbruggen et. al., “Function and failure of the fetal membrane: Modelling the mechanics of the chorion and amnion” PLOS ONE Mar. 28, 2017). The amnion provides some heft to the chorion to ensure the dual layer amnion-chorion inserts are user friendly enough for applying the insert to the anterior surface of the eye.

Single layer amnion membranes are especially effective given how thin the membranes are, which improves patient comfort and tolerability. The amnion membranes contain native bioactive factors that are suitable for delivery to the ocular surface. The bioactive factors are particularly suitable for delivery directly onto the cornea, and are known to modulate inflammation and fibrosis to promote wound healing and provide antimicrobial properties. However, single layer amnion membranes are limited in terms of dosing of the bioactive factors that can reach the ocular surface over a reasonable therapeutic time-frame. Amnion-chorion membranes have more bioactive factors (e.g., 4-5 times as many bioactive factors) compared to amnion alone. The thickness of amnion-choroid membranes or any multi-layer construct is typically greater than the single layer constructs, which can reduce patient comfort and tolerability due to foreign body sensations. Described herein are multi-layer constructs including a dual layer of amnion-chorion, chorion-amnion, amnion-amnion, or chorion-chorion, a triple layer of amnion-amnion-amnion, chorion-amnion-amnion, amnion-chorion-amnion, amnion-amnion-chorion, chorion-chorion-amnion, amnion-chorion-chorion, chorion-amnion-chorion, or chorion-chorion-chorion, a quadruple layer of amnion-chorion-chorion-amnion or chorion-amnion-amnion-chorion, as well as another other multilayer constructs of the amnion and chorion. Preferably, the amnion layer is against the anterior surface of the eye and the chorion layer is sandwiched in between amnion layers. The multi-layer constructs described herein provide maximal bioactive factor delivery to the eye and are constructed to minimize foreign body sensations thereby improving patient tolerability.

The biomaterial can include or be capable of releasing one or more bioactive factors of the biomaterial. As used herein, “bioactive factor” means a factor natively generated by and/or contained in a biomaterial that provides a biological effect, including growth factors, cytokines, chemokines, and the like. The bioactive factor can provide additional therapeutic benefit for a disease or condition by promoting healing, reducing inflammation, and the like. For example, the biomaterial can be a tissue that contains and/or releases healing factors naturally-occurring in and/or derived from the tissue that have anti-fibrotic, anti-inflammatory, anti-neovascular effects, or the like for repair and regeneration at the site of implantation or near the site of implantation. The biomaterial can be whole amniotic membrane that contains and/or releases one or more bioactive factors, including regenerative and anti-fibrotic factors that aid in the control of inflammation and scarring including, but not limited to ANG1 (Angiopoietin 1), bFGF (Basic Fibroblast Growth Factor), EGF (Epidermal Growth Factor), G-CSF (Granulocyte Colony-Stimulating Factor), GDF11 (Growth Differentiation Factor 11), HGF (Hepatocyte Growth Factor), HC-HA/PTX3 (Heavy-Chain Hyaluronan/Pentraxin 3 proteoglycan complex), IL-1B (Interleukin 1B), IL-4 (Interleukin-4), IL-6 (Interleukin-6), IL-8 (Interleukin-8), IL-10 (Interleukin-10), NGF (Nerve Growth Factor), PDGF-AA (Platelet Derived Growth Factor AA), PDGF-BB (Platelet Derived Growth Factor BB), PGE2 (Prostaglandin E2), PIGF (Placental Growth Factor), SDF-1a (Stromal cell-derived factor 1a), TIMP-1 (Tissue Inhibitor of Metalloproteinases-1), TIMP-2 (Tissue Inhibitor of Metalloproteinases-2), TIMP-4 (Tissue Inhibitor of Metalloproteinases-4), TGF-alpha (Transforming Growth Factor Alpha), TGF-beta (Transforming Growth Factor Beta), TSP1 (Thrombospondin-1), VEGF (Vascular Endothelial Growth Factor), VEGF-R (VEGF Receptor), and WNT4 (Wingless-Type MMTV Integration Site Family, Member 4). The amniotic membrane contains both growth promoting, proteins that regulate inflammation, and proteins that exhibit antimicrobial properties and growth inhibiting proteins (see, e.g., Clinical Ophthalmology 2019:13, 887-894).

The biologically-derived material can be amniotic membrane. The amniotic membrane tissue can be derived from amniotic sac of the placenta. The amniotic sac surrounding a fetus includes the amnion and chorion, both derived from the inner layer of the placenta. The amniotic membrane is part of the amnion and includes a layer of epithelial cells, a basement membrane, and an avascular stromal matrix. Amniotic membrane contains pluripotent cells, highly organized collagen, anti-fibrotic and anti-inflammatory cytokines, immune-modulators, growth factors, and matrix proteins and can promote healing in eyes (Murri et. al. Clin. Ophthalmol. (2018) 12:1105-1112). The biomaterial can be of hydrophilic or hydrophobic nature. The biomaterial can include or be impregnated with one or more therapeutic agents for additional treatment of a disease process.

While inserted on the ocular surface or within a punctal aperture, the amniotic membrane can release factors useful for tissue repair and regeneration. The amniotic membrane can bio-absorb after at least about 1 week up to about 3 to 6 months. The inserts formed of amniotic membrane can be placed on the ocular surface and/or within a punctal aperture for a fixed period of time for at least 1 day up to about 3 to 6 months and then removed before bio-absorption, after at least one bioactive factor has completely or partially diffused into the tear film. The inserts formed of amniotic membrane can be used for delivery of beneficial diffusible factors from the tissue that can be helpful against a variety of conditions. The inserts formed of amniotic membrane can be inserted alone as a primary treatment or in combination with another biotissue implant(s) formed of other materials (e.g., sclera) or with another medical therapy (e.g., eye drops composed of one or more drugs) as an adjunct anti-fibrotic or anti-inflammatory treatment.

Regardless of the source or type of biomaterial used, the biomaterial can be minimally manipulated or minimally modified. The minimal structural modification of the biomaterial for use in or on a patient can include being freeze-dried, lyophilized membrane, dehydrated, cryopreserved, or otherwise minimally-manipulated prior to implantation (see, e.g., SURGRAFT dehydrated amniotic sheets, or SURSIGHT ocular amniotic membrane allograft, Surgenex Scottsdale, AZ, PROKERA). Minimal modification also includes cutting the biomaterial into a three-dimensional shape or shapes other than a naturally-occurring three-dimensional shape of the biomaterial, including disks, plugs, pellets, rods, strips, beads, annulus, or other shapes.

The bio-tissue insert of amniotic membrane can be used alone as a primary treatment or together with an adjunct treatment. The bio-tissue insert of amniotic membrane can be positioned at any of a variety of locations including outside the cornea against the sclera, fornix, bulbar conjunctival surface, cul-de-sac or other part of the eye. The bio-tissue insert of amniotic membrane can be positioned within one or both punctal apertures of an eye.

The inserts described herein are preferably formed of biologic material (e.g., tissue) and are self-retained in the eye in a sutureless manner. In some implementations, the inserts are retained without any support or retention aids provided by non-biologic material. Non-biologic material includes synthetic materials prepared through artificial synthesis, processing, or manufacture that may be biologically compatible, but that are not cell-based or tissue-based. For example, non-biologic material includes synthetic polymers, copolymers, polymer blends, and plastics. Non-biologic material includes inorganic polymers such as silicone rubber, polysiloxanes, polysilanes, and organic polymers such as polyethylene, polypropylene, polyvinyls, polyimide, etc. The inserts formed of biologic material can be combined with biodegradable or non-biodegradable hardware materials for additional structural stability. The insert formed of biologic material can be cryopreserved, dehydrated, lyopreserved, or non-preserved. The insert can be sterilized prior to use.

Regardless the source or type of biologically-derived material, the material can be cut into a shape and/or formed into an insert suitable for positioning on the ocular surface. The form factor of the cut inserts can vary as described elsewhere herein. For example, the tissue can be formed into a disk, pellet, plug, or rod shape or a closed loop shape for positioning against the ocular surface to provide release of healing factors from the tissue. The size and shape of the insert can vary depending on the target location as will be described in more detail below.

The material of the insert, which is preferably formed of a biological tissue, can naturally contain a certain level of biologic factors that are relatively homogeneously distributed within the material. The size range of the insert can be selected to ensure wearability and comfort, ease of use and handling by clinicians applying the insert to a patient's eye, as well as minimum cubic volume so the insert is capable of eluting biologic factors to the eye within a therapeutic range and/or for biologic activity. The wearability can be provided by a minimum inner diameter of the insert so avoid contact between the insert 100 and the cornea (e.g., 12 mm inner diameter). The wearability can also be provided by a maximum outer diameter that is not too big (e.g., 40 mm outer diameter, preferably about 20 mm-30 mm, more preferably about 22 mm-27 mm) and a width or length that is easy to handle by a clinician placing the insert on the eye, and a width or length that is not too big to induce foreign body sensation. The minimum cubic volume of the insert 100 in the shape of a ring can be at least about 1 mm³, 2 mm³, 3 mm³, 4 mm³, 5 mm³, 6 mm³, 7 mm³, 8 mm³, 9 mm³, 10 mm³, 11 mm³, 12 mm³, 13 mm³, 14 mm³, 15 mm³, 16 mm³, 17 mm³, 18 mm³, 19 mm³, 20 mm³, 21 mm³, 22 mm³, 23 mm³, 24 mm³, 25 mm³, 50 mm³, 75 mm³, 100 mm³, 125 mm³, 150 mm³ or greater. The minimum cubic volume of the insert 100 can be about 1 mm³ to about 150 mm³, preferably about 14 mm³.

For inserts in a ring configuration, the difference between the inner diameter and the outer diameter (i.e., width of the insert), as well as the thickness of the insert between the anterior surface to the posterior surface of the insert contributes to the total cubic volume of the insert. The width of the ring provides ease of use to the insert—given that a clinician, such as an ophthalmologist may apply the insert in an operative room or clinic or office setting. A minimum width of about 0.5 mm reliably allows ease of handling and insertion/placement of the insert onto the ocular surface. The minimum width can provide structural rigidity sufficient for manual manipulation by a clinician or other person who is performing the insertion procedure, while also providing a minimal foreign body sensation to the patient. The insert made of amniotic tissue can be positioned on the eye using a finger(s) and with the assistance of a cotton swab to adjust its position under topical anesthesia or with no anesthesia. The insert is applied to the eye so no folding or overlap of the membrane occurs and the lower surface of the membrane lies flush against the anterior surface of the eye. As described elsewhere herein, where the insert is in a shape of a ring the size of the ring can vary. The inner diameter of the insert 100 is larger than a patient's cornea so that it encircles with the cornea without touching the cornea, for example, greater than about 9 mm or about 15 mm to accommodate a wider range of corneal sizes. The insert made of amniotic tissue is nearly invisible once positioned on the eye and the tissue blends in with the conjunctiva. The insert can be removed depending on the degree of inflammation in the eye being treated and total time positioned against the eye. The insert may also be completely absorbed within the eye upon positioning for a time.

Processing of chorioamniotic and amniotic membrane tissue for clinical use (e.g., cryopreservation, dehydration, etc.) can lead to variability in thickness that, together with the natural variability in thickness of the tissues, impacts overall cubic volume of the insert formed from the tissue. The insert geometry is designed to achieve a cubic volume that takes into account the natural and acquired variabilities of the tissue during tissue processing. In some implementations, the cubic volume can be within a range of at least about 100 mm³. The insert geometry can achieve a cubic volume that falls within a range of at least 1 mm³ up to about 150 mm³. The upper limit can be about 200 micron thickness by an inner diameter of about 12 mm and an outer diameter of about 33 mm.

It should be appreciated that the minimum cubic volume of the insert 100 can be outside this range, but the biologic activity would decrease or increase accordingly. Similarly, if the insert 100 is formed of a dual layer of amnion and chorion, more biologic activity may be observed compared to biologic activity of an insert 100 formed of single layer amnion. A study measuring cytokines in allografts found multilayer amnion-chorion contained similar amounts of each factor measured when normalized per dry weight. However, single layer amnion allografts contained on average only 25% as much of each factor as the chorion when calculated per surface area of tissue applied to a wound (see Koob et. al., “Cytokines in single layer amnion allografts compared to multilayer amnion/chorion allografts for wound healing” J. Biomed Mater Res B Appl. Biomater. 2015 Jul; 103(5):1133-40).

While the total volume predicts the overall potential loading of bioactive factors, the precise thickness has variability due to the natural variation in human tissues, both naturally as well as post-processing (e.g., dehydration, cryopreservation, lyophilization, etc.). The pre-processed thickness may also differ from the post-processed, rehydrated thickness, due to changes in the underlying structure of the membrane due to its processing. Therefore, the surface area constructed out of a two-dimensional amniotic membrane sheet, as well as selection of layers (single layer amnion, dual or multilayer amnion, amnion-chorion, dual or multilayer amnion-chorion, etc.), will be a determining factor in the overall volume of the construct. In one embodiment, a single layer of amnion-chorion tissue, combined with ring width dimensions described above (a minimum of 1-2 mm in width), with an inner diameter of at least 12 mm (larger than an average cornea), provides adequate structural integrity and rigidity for manual placement of the ring in the eye, as well as adequate volume/surface area for bioactive factor dosing.

The insert can be a single layer of amnion tissue, a multi-stack amnion layer, a single or multi-stack amnion-chorion tissue having an outer diameter that is about 15-35 mm, preferably about 17-33 mm, an inner diameter that is about 12-30 mm, preferably about 16-28 mm, a total area of about 30-1000 mm², a width of about 0.5-6 mm, a thickness of about 0.02-0.5 mm, and a total volume that is about 1-150 mm³, preferably about 5-50 mm³, more preferably about 10-20 mm³.

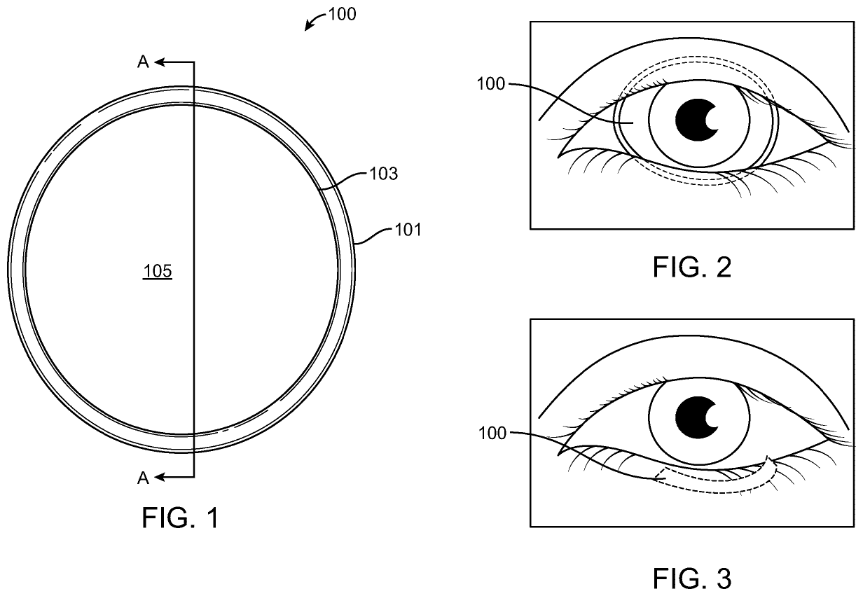

FIG. 1 illustrates an implementation of an insert 100 made of bio-tissue that is configured for self-retaining, sutureless, clear-cornea application to an ocular surface. The insert has an outer edge 101 and an inner edge 103, inside of the outer edge 101. The inner edge 103 defines an opening or window 105. The window 105 is sized to extend around a circumference of a cornea to provide for a clear visual axis and to avoid contact with the highly-sensitive corneal surface. The window 105 can be formed by an opening or cutout in the bio-tissue material forming the insert 100 or by forming a closed loop from an elongated strip of material, which is described elsewhere herein. The inner diameter of the insert 100 (i.e., inner diameter of the inner edge 103) is at least about or greater than 9 mm, or at least about 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, and 30 mm. The ocular amniotic peripheral ring can have an outer diameter (i.e., diameter of the outer edge 101) sized so at least a portion of the insert 100 can be positioned on the bulbar conjunctival surface under both eyelids (see FIG. 2). The outer diameter of the insert can vary depending on the inner diameter of the insert. The insert 100 is preferably positioned on a region of the anterior surface of the eye that is outside an optical zone of the eye so that the insert is out of contact with the cornea. Preferably, the outer diameter of the insert is no greater than about 40 mm, 39 mm, 38 mm, 37 mm, 36 mm, 35 mm, 34 mm, 33 mm, 32 mm, 31 mm, 30 mm, 29 mm, 28 mm, 27 mm, 26 mm, 25 mm, 24 mm, 23 mm, 22 mm, 21 mm, and 20 mm and is at least about 12 mm to conform to the bulbar conjunctiva of eye. This outer diameter ensures that the superior part of the insert is retained under the upper eyelid and inferior part of the insert is retained in the cul-de-sac of the lower eye lid, while the medial and lateral sides are retained within the canthi of the eye. In a preferred implementation, the inner diameter of the insert 100 defined by the inner edge 103 is about 20 mm and the outer diameter of the insert 100 defined by the outer edge 101 is no greater than about 30 mm.

In an implementation, the insert 100 has an inner diameter of about 20-22 mm, an outer diameter of about 24-27 mm, a thickness between upper and lower surfaces that is about 0.05-0.150 mm, and a volume that is about 10-18 mm³. Thickness of the insert can vary due to anatomy, however, a nominal thickness of 0.1 mm provides a volume of about 14 mm³. The dimensions of this implementation provide a comfortable fit for most eyes and ensures the insert avoids touching the cornea, and avoids buckling or folding, such as at the lateral canthus, which can impact retention and comfort. The dimensions of this implementation also make the insert easy for handling by the clinician applying the insert to the ocular surface, which can be applied manually and adjusted with a cotton swab or similar instrument. The dimensions of this implementation also provide a total surface area / volume sufficient to achieve bioactive factor dose to the patient from the insert 100 for anti-fibrotic, anti-inflammatory, or anti-neovascular therapeutic effect while positioned on the eye surface.

The insert can have a width between the outer edge 101 and the inner edge 103. The width can be at least about 100 microns and no greater than about 8 mm, no greater than about 7 mm, no greater than about 6 mm, no greater than about 5 mm, no greater than about 4 mm, no greater than about 3 mm, no greater than about 2 mm, or no greater than about 1 mm. Preferably, the width between the outer edge 101 and the inner edge 103 is about 0.5 mm up to about 16 mm, preferably 1 mm up to about 5 mm. The total area of the annulus formed by the insert can be about 30 mm² up to about 1000 mm², preferably about 50 mm² to about 200 mm².

The insert can have a thickness between the lower surface of the ring (i.e., the portion contacting the anterior surface of the eye) and the upper surface that is at least 15 microns up to about 800 microns, preferably the thickness is about 20-200 microns depending on whether the implant is a single layer amnion, multi-layer amnion, single layer amniochorion or multi-layer amniochorion.

In other implementations, the minimal structural modification of the biological tissue includes cutting the tissue into a first shape and then forming the insert into a second shape. In some implementations, the first shape is a disc shape and the second shape is a closed loop. The disc shape can have an outer diameter as described above, for example, no greater than about 40 mm or another size that is shaped to fit on a surface of an eye. The disc can be cut to have an inner diameter sized large enough to ensure the bio-tissue can remain out of contact with the cornea (e.g., at least about 8-9 mm). The disc shape can be a regular geometric shape (e.g., circular, oval, elliptical, polygonal, and the like) or the disc shape can be a more irregular shape (e.g., fusiform) that is then cut into an annular shape as discussed above. The geometry of the insert 100 can vary. In some implementations, the insert 100 includes a connected region forming a closed loop having any of a variety of shapes, including round, such as a circular, oval, elliptical shape. The geometry of the closed loop can also include serpentine, saddle shape, toric, or polygonal having n number of sides. The geometry may be a polygon, including but not limited to a rectangle, pentagon, hexagon, octagon, etc. The geometry may be a combination of circular and straight segments, such as a half-circle. The geometry may include a variable width along the circumference of the insert, including but not limited to flaps, pads, wings, in order to increase total surface area/volume and thereby increase the bioactive factor dose provided by the insert. These features may particularly be on the superior or inferior portions of the insert, resting underneath the eyelid, to avoid the lid margins or the region where the eyelids meet when the eye closes or blinks.

The minimal structural modification of the tissue can adjust a size of the tissue to form an insert 100 having a form factor that is an elongate pellet, rod, strip, ring segment or other elongate insert sized and shaped for positioning on an ocular surface, such as under one or both eye lids. In this manner, the bio-tissue insert 100 need not form a closed loop shape and instead can be positioned on the eye out of contact with the cornea, but as an elongate strip or segment. FIG. 3 illustrates an ocular amniotic peripheral insert segment positioned on the bulbar surface under the lower eye lid. The insert can have an outer edge, an inner edge, a first free end, and a second free end opposite the first. The insert can have length between the first free end and the second free end, a width between the outer edge and the inner edge, and a thickness. The length can be about 5 mm and preferably no greater than about 33 mm. The width can be about 1 mm, preferably no greater than about 100 mm. The thickness, which can be the thickness of the starting biological material (i.e., amniotic membrane), can be no greater than about 5 mm. Amniotic membrane is typically between 0.02 mm-0.05 mm. In an implementation, the insert 100 can include one or more ring segments, strips or elongated inserts that are not wider than 10 mm and longer than 30 mm. The insert 100 can be inserted in the cul-de-sac of the lower lid or underneath the upper lid on the sub-palpebral ocular surface, or both.

Creating a ring shape from a disc shape of biologic tissue generates a large surface area of tissue that remains unused and thereby increases the cost to manufacture the insert. In some implementations, the minimal structural modification of the biological tissue (e.g., amniotic membrane) can include a longitudinal cutting into an elongate strip of tissue that is then formed into another shape, such as a closed loop shape. The elongate strip, for example, can be sized so that upon forming a closed loop forms an insert 100 having an inner diameter as described above. Generally, the elongate strip of tissue has a width that is less than its length.

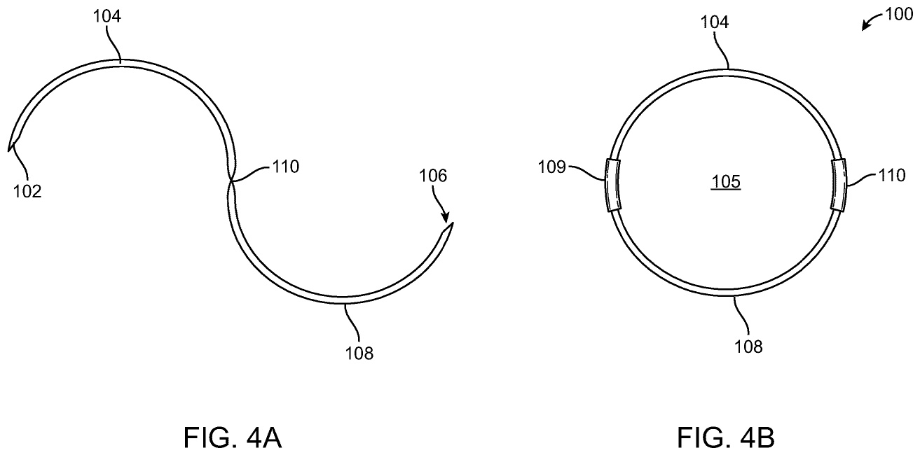

Manufacturing the inserts using bio-tissue cut into strips that are later fused into a closed loop ensures nearly all tissue is used with less waste. FIG. 4A illustrates an initial cut form factor of bio-tissue having a thin sine wave shape. The strip of bio-tissue has a first segment 104 having a first free end 102. The first segment 104 has an arc shape. The strip of bio-tissue has a second segment 108 having a second free end 106. The second segment 108 has an arc shape corresponding to the shape of the first segment 104. The first segment 104 and the second segment 108 are twisted relative to one another at a hinge point 110 that is about half-way between the first free end 102 and the second free send 106. The arc shapes and the twisted hinge point 110 creates a sine wave shape of the initial cut form factor. FIG. 4B illustrates the bio-tissue insert 100 formed by fusing the first free end 102 and the second free end 106 together at a first joint 109 forming a closed loop. The hinge point 110 where the segments 104, 108 are twisted can be fixed forming a second joint 111. The joints can be formed by a variety of fixation methods, including a polymer coating, adhesive, or other material. The fixation can alternatively be done without any additional materials, but using the processing of the amniotic membrane itself, e.g., cutting the tissue in its wet, unprocessed form, then clamping the structure in place during processing (e.g., dehydration, cryopreservation, lyophilization, etc.). The first joint 109 and the hinge point 110 ensure the insert 100 maintains the closed loop and the arc shapes of the segments 104, 108 result in a substantially circular shape to the insert 100.

The joint 109 can be formed by adhering the ends, such as by wetting dehydrated tissue allowing them to stick together. In some implementations, the joint 109 is formed by mechanically linking the first and second free ends 102, 106 together. For example, each of the free ends can have a partial slit formed through its width that is sized to receive the partial slit of the opposite end.

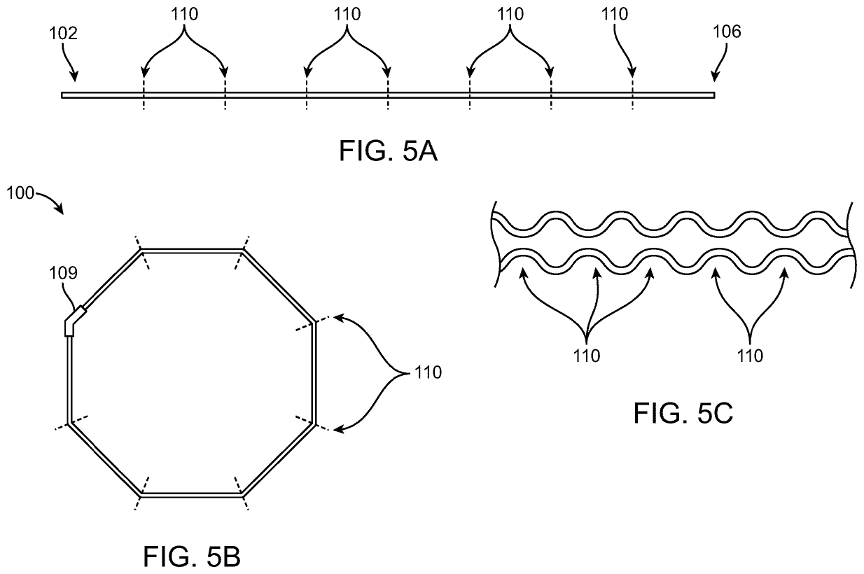

The closed loop insert need not have a smooth circular shape as shown in FIG. 1 or FIG. 4B. The insert 100 can have a closed shape having n sides forming a polygon. FIG. 5A illustrates an initial cut form factor of bio-tissue having a thin elongate shape. The strip of bio-tissue has a first free end 102 and a second free end 106. FIG. 5B illustrates the bio-tissue insert 100 formed by folding the strip of FIG. 5A at multiple hinge points 110 along its length and fusing the first free end 102 and the second free end 106 together at a single joint 109 forming a closed loop. Unlike the closed loop shape of FIG. 4B, the closed loop shape of FIG. 5B incorporates multiple sides forming a polygonal closed loop. The number and angle of the hinge points 110 can vary. The hinge points 110 can have an angle that varies depending on the closed shape having n sides. The insert 100 shown in FIG. 5B is created by folding the strip of FIG. 5A at 8 hinge points 110 thereby forming an 8-sided polygon (i.e., octagon). Thus, the angle between sides at each hinge point 110 is 135 degrees.

The hinge points 110 can incorporate features to facilitate folding of the bio-tissue strip, such as slits in the bio-tissue at the desired location of the hinge points 110. The bio-tissue can incorporate a narrower width at the desired hinge points 110 to encourage folding. The strip of bio-tissue need not be straight along its entire length prior to folding and can instead be cut into an elongate strip having undulating edges (see FIG. 5C). The undulating edges can aid in folding the elongate strip into a closed loop having a smoother shape. The pattern of the undulations on each edge can mirror one another such that each edge has a smoothly falling form towards its opposing edge creating a narrowing region for the hinge points 110 and a smoothly rising form away from one another creating a widening region between the hinge points 110 (see FIG. 5C). In still further implementations, the bio-tissue can be formed into another three-dimensional shape such as a ribbon shape that is then wrapped around a cylinder or cone to flex the bio-tissue insert before being placed onto the eye. Whether the bio-tissue is cut into an annulus, an S-shaped strip, a straight strip, or an undulating strip, the bio-tissue insert is prepared in a minimally-modified manner with cutting, shaping, folding, bending, and fusing, etc.

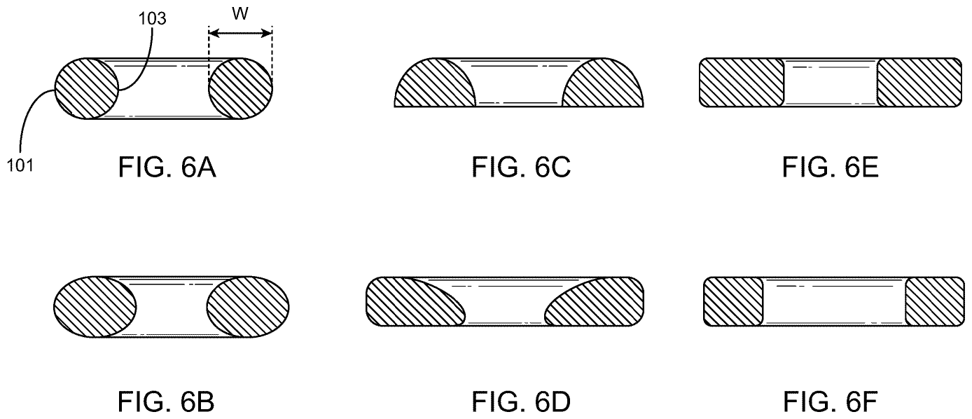

The cross-section of the bio-tissue insert 100 (i.e., cross-section taken from a surface of the tissue configured to be placed against the eye to a surface of the tissue that is configured to face away from the surface) can have different shapes. The shape of the cross-section can be rounded or angular. For example, the rounded cross-sectional shape can include a circle, oval, ellipse, hemi-circle, and the like. The angular cross-sectional shape can include rectangular or square shapes having at least one flat surface, akin to a flat sheet. The cross-section of the insert 100 can have a width W between the inner edge 103 and the outer edge 101 that is at least about 0.1 mm to about 10 mm, preferably about 1 mm up to about 5 mm. FIGS. 6A-6F illustrate example cross-sections of the insert 100 of FIG. 1 taken at line A-A including circular (FIG. 6A), oval (FIG. 6B), hemi-spherical (FIG. 6C), and tear-drop shaped (FIG. 6D), rectangular (FIG. 6E), square (FIG. 6F) and the like.

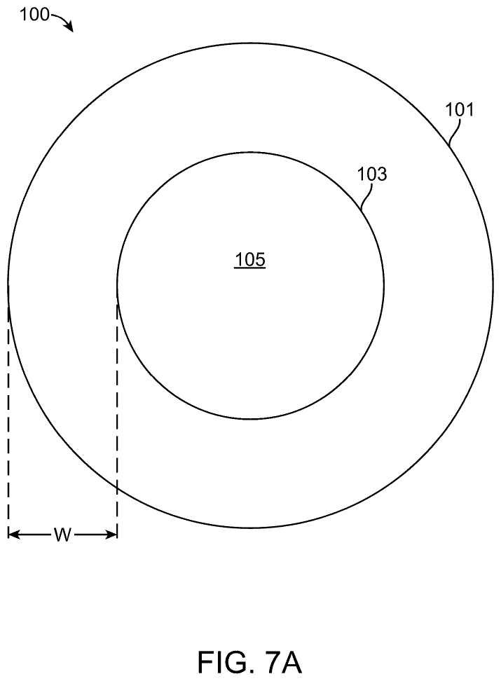

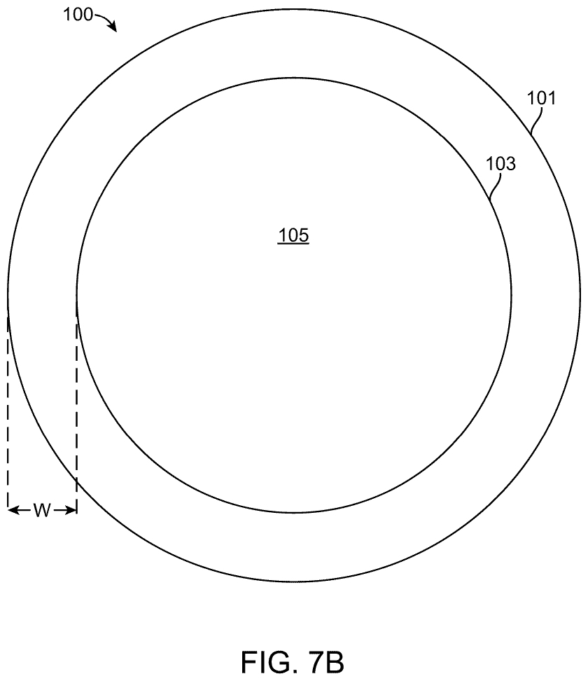

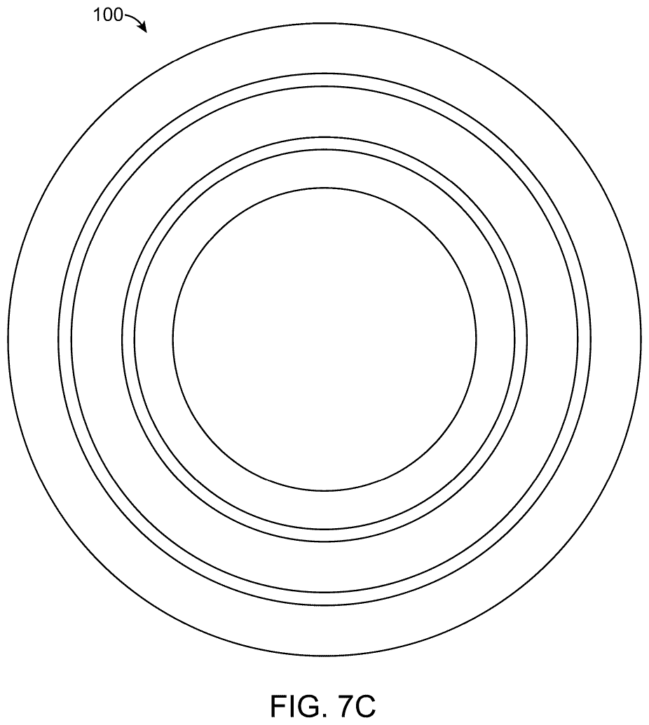

As discussed above, the bio-tissue insert 100 can be in the shape of a closed loop of any of a variety of shapes. FIGS. 7A-7C illustrate various implementations of a bio-tissue insert 100 having substantially circular shapes.

FIG. 7A illustrates an implementation of an insert 100 formed of bio-tissue that is amnion-chorion in a substantially annular shape. The insert 100 has an outer edge 101 and an inner edge 103, inside of the outer edge 101. The inner edge 103 defines an opening or window 105 that is sized to extend around a circumference of a cornea to provide for a clear visual axis and to avoid contact with the highly-sensitive corneal surface. The window 105 can be formed by an opening or cutout in the bio-tissue material forming the insert 100 or by forming a closed loop from an elongated strip of material, which is described elsewhere herein. The inner diameter and outer diameter of the insert 100 can vary as described elsewhere herein. In an example, the inner diameter is 11 mm and the outer diameter is 20 mm providing a width W that is about 4.5 mm and a volume of 22 mm³, assuming a thickness of about 100 microns. FIG. 7B illustrates another implementation of an insert 100 formed of amnion-chorion that is substantially annular in shape. The inner diameter and outer diameter shown in FIG. 7B are larger than what is shown in FIG. 7A. The inner diameter is about 19 mm and the outer diameter is about 25 mm providing a width W that is about 3 mm and a volume of about 21 mm³, assuming a thickness of about 100 microns. The dimensions provided are merely examples and are not intended to be limiting. The inner diameter, outer diameter, thickness, and volumes can vary as described elsewhere herein.

FIG. 7C illustrates an implementation of a kit of concentric bio-tissue inserts 100. The concentric inserts 100 can be cut into shape from a single piece of bio-tissue, including a single layer of amnion or chorion, or a multi-layer construct of amnion and chorion layers. The inserts 100 forming the kit can be provided together to the clinician, for example, on a backing paper within a sterile package, who then selects one insert 100 from the plurality of bio-tissue inserts 100 within the kit that has a desired size and shape for positioning on an ocular surface of the patient based on the patient's ocular anatomy and comfort level. Each dark line in FIG. 7C represents a cut such that the kit includes a first insert having an inner diameter and an outer diameter that are both smaller than the second insert and the second insert has an inner diameter and an outer diameter that are both smaller than the third insert. The cut for the outer diameter of the first insert is shown as a different cut from the one forming the inner diameter of the second insert and the cut for the outer diameter of the second insert is shown as a different cut from the one forming the inner diameter of the third insert. However, it should also be appreciated that the cuts for the inner diameter of one insert can be the same cut as the one for the outer diameter of another insert. The shape of the inserts 100 shown in FIG. 7C are circular rings, but it should be appreciated that the inner dimension and outer dimensions of the inserts 100 can vary as described elsewhere herein, including various non-circular shapes.

One or both of the window 105 defined by the inner edge 103 and the outer edge 101 can be substantially circular or substantially non-circular in any of the bio-tissue inserts 100 described herein. For example, the outer edge 101 can be non-circular and the inner edge 103 can be non-circular. The outer edge 101 can be circular and the inner edge 103 can be circular. The outer edge 101 can be circular and the inner edge 103 can be non-circular. The outer edge 101 can be non-circular and the inner edge 103 can be circular.

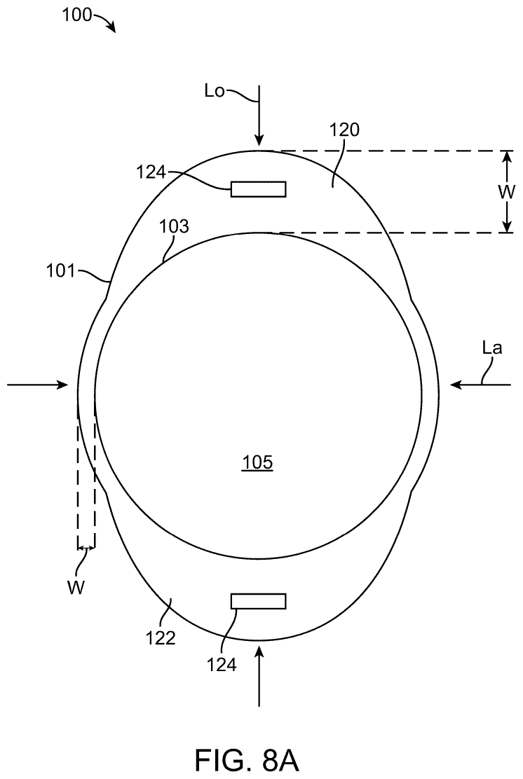

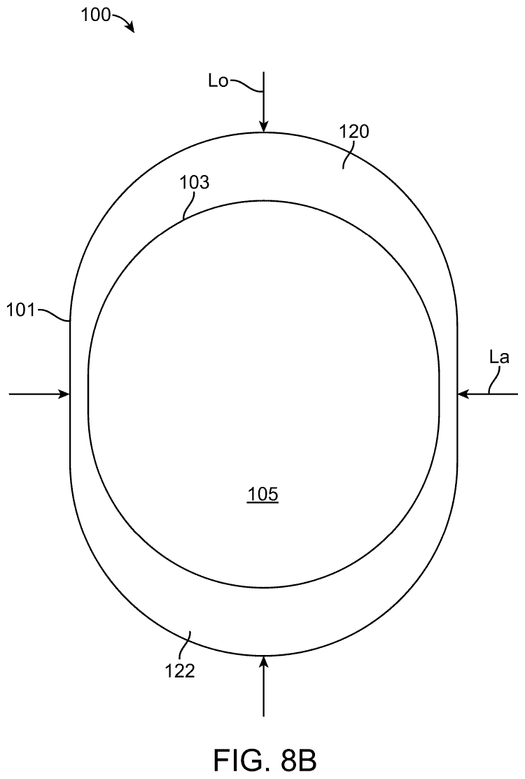

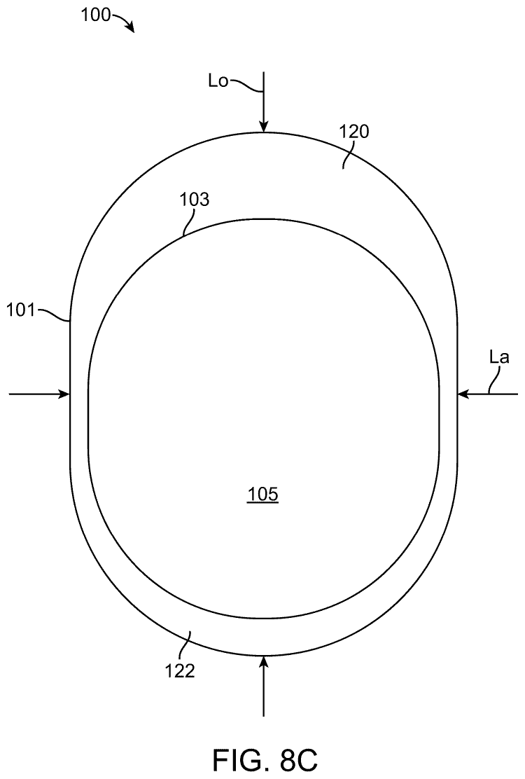

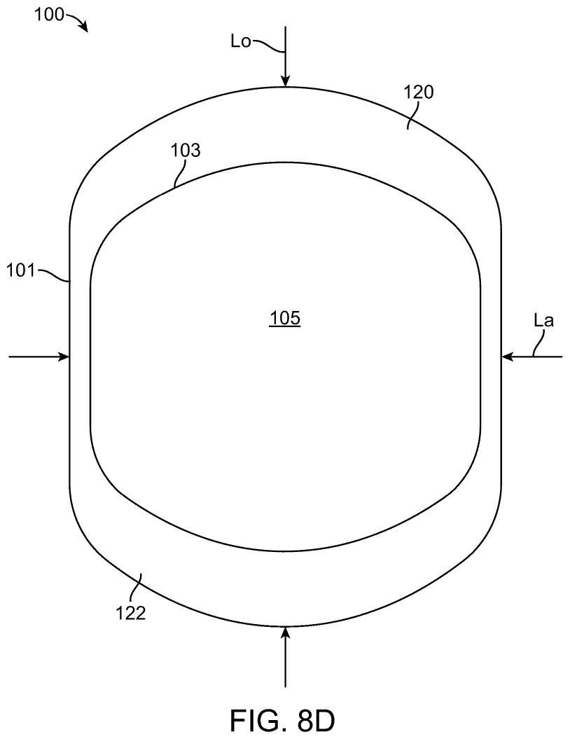

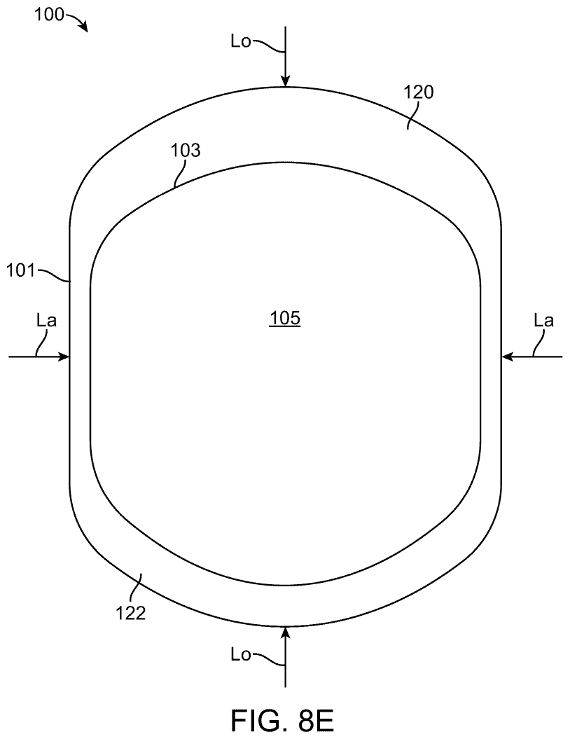

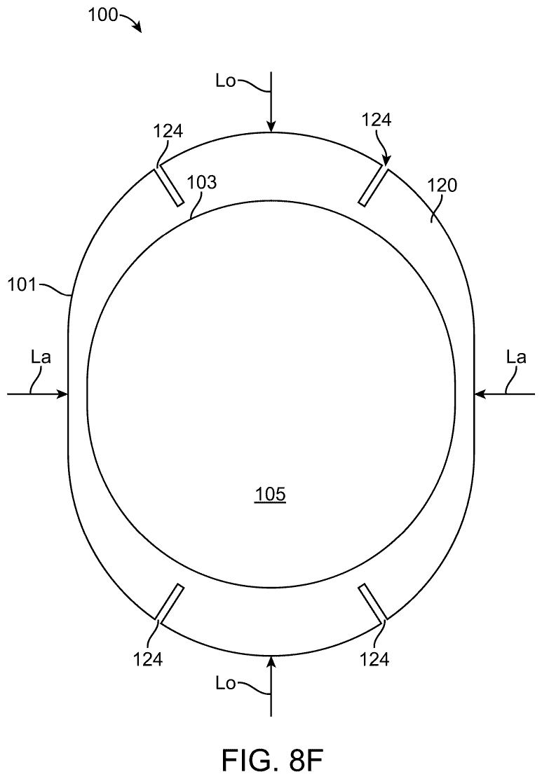

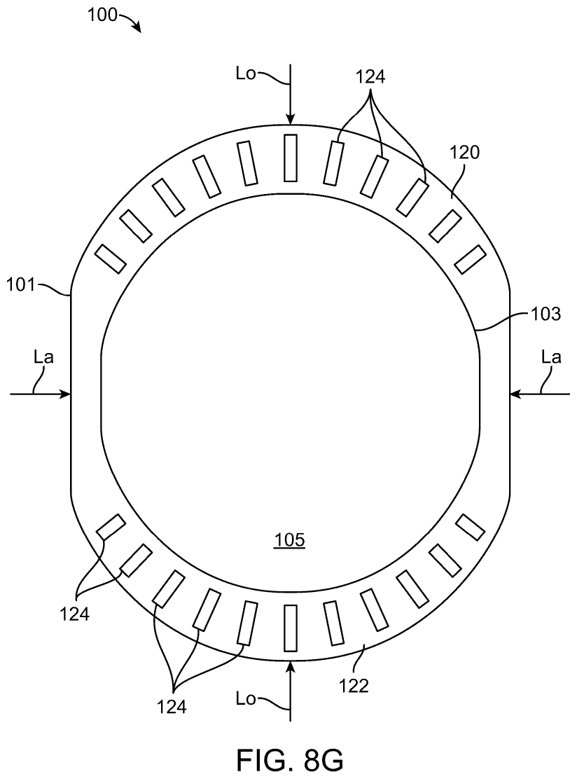

FIGS. 8A-8G illustrate various implementations of bio-tissue inserts 100 having substantially non-circular outer perimeter. The non-circular outer perimeter shape can vary including lentoid, oval, elliptical, pill shape, discorectangle, obround, stadion, egg, quatrefoil, and the like. The lateral dimension of the non-circular insert 100 (i.e., dimension that is side-to-side) is preferably less than the longitudinal dimension of the non-circular insert 100 (i.e., dimension that is up-and-down). Thus, the maximum dimension across the insert 100 is the longitudinal dimension so that the insert is taller than it is wide. This orientation of the non-circular insert 100 inhibits contact with the caruncle located nasally near the medial canthus of the eye and the opposite lateral canthus, which can contribute to foreign body sensations. An insert 100 that is too wide can fold or bunch up near these locations. However, the subpalpebral space, both superiorly and inferiorly, has more space for receiving the insert 100.

FIG. 8A illustrates an implementation of an insert 100 formed of a dual layer of amnion-chorion. The insert 100 has an outer edge 101 and an inner edge 103, inside of the outer edge 101. The inner edge 103 defines an opening or window 105 that is substantially centrally placed relative to the insert 100 overall. The window 105 defined by the inner edge 103 can be substantially circular in shape whereas the shape of the insert 100 overall defined by the outer edge 101 is substantially non-circular. The window 105 is sized to extend around a circumference of a cornea to provide for a clear visual axis and to avoid contact with the highly-sensitive corneal surface. The insert 100 is illustrated as it would appear positioned on an eye with a maximum longitudinal dimension Lo greater than a maximum lateral dimension La. The maximum longitudinal dimension Lo can be about 15 mm-35 mm, preferably about 25 mm-29 mm, and the maximum lateral dimension La can be about 15-25 mm, preferably about 20 mm. The inner diameter of the insert 100 defined by the inner edge 103 can be about 14 mm-20 mm, preferably about 18 mm.

The shape of the outer edge 101 provides the insert 100 with a first lobe 120 located on an upper end of the insert 100 along the maximum longitudinal dimension Lo and a second lobe 122 located on a lower end of the insert along the maximum longitudinal dimension Lo. The upper and lower lobes 120, 122 provide discrete regions around the circumference of the insert 100 of enlarged widths W between the inner edge 103 and the outer edge 101 that are arranged to be positioned under the lids away from the lid margins. The greater width W of the upper and lower lobes 120, 122 relative to the more centrally-located sides increases friction and prevents potential slippage of the insert 100 relative to the eye surface. The widths W of the upper and lower lobes 120, 122 between the inner and outer edges also increases the total bioactive factor load such that the insert 100 can deliver sufficient bioactive factors to the ocular surface and the tear film. In the implementation of FIG. 8A, the lobes 120, 122 are narrower laterally compared to the maximum lateral dimension La near the center of the insert. For example, the width of the lobes can be about 15 mm whereas the maximum lateral dimension La can be about 20 mm.

FIG. 8B illustrates an implementation of an insert 100 formed of a dual layer of amnion-chorion. The insert 100 has an outer edge 101 and an inner edge 103, inside of the outer edge 101. The outer edge 101 defines a substantially non-circular shape to the insert 100. The insert 100 is illustrated as it would appear positioned on an eye with a maximum longitudinal dimension Lo greater than a maximum lateral dimension La. The maximum longitudinal dimension Lo can be about 15 mm-35 mm, preferably about 25 mm-29 mm, and the maximum lateral dimension La can be about 15 mm-25 mm, preferably about 20 mm. The inner diameter of the insert 100 defined by the inner edge 103 can be about 14 mm-25 mm, preferably about 18 mm-20 mm, with the internal hole geometry substantially non-circular. The inner edge 103 defining the window 105 is also substantially non-circular in shape and has a greater height than its width (although it can optionally be circular as described elsewhere herein). The window 105 defined by the inner edge 103 can be substantially centrally positioned relative to the rest of the insert 100 or asymmetrically positioned relative to the insert 100. The inner diameter of the insert 100 along the longitudinal dimension is about 20 mm and the inner diameter of the insert 100 along the lateral dimension La is about 18 mm. The insert 100 can include two lines of symmetry such that the right and left sides of the insert 100 on either side of the longitudinal line Lo are the same and the upper and lower portions of the insert 100 on either side of the lateral line La are the same.

As with the implementation shown in FIG. 8A, the insert 100 of FIG. 8B has a first lobe 120 located on an upper end of the insert 100 along the maximum longitudinal dimension Lo and a second lobe 122 located on a lower end of the insert along the maximum longitudinal dimension Lo. The upper and lower lobes 120, 122 provide discrete regions around the circumference of the insert 100 of enlarged widths W between the inner edge 103 and the outer edge 101 that are arranged to be positioned under the lids away from the lid margins. The greater width W of the upper lobe 120 can, but need not be the same as the size of the lower lobe 122. The upper and lower lobes 120, 122 have a width W between the inner edge 103 and the outer edge 101 that is greater than the width at the sides. For example, the lobe 120 on the upper end of the insert 100 can be about 2.5 mm to about 3.5 mm wide between the inner edge 103 and the outer edge 101 whereas the width W between the inner edge 103 and the outer edge 101 at the more centrally located lateral sides of the insert 100 can be narrower at about 1 mm wide. The upper and lower lobes 120, 122 can have the same width W as one another or can have a different width W as one another such that the first lobe 120 on the upper end can be narrower or wider than the second lobe 122 at the lower end of the insert along the maximum longitudinal dimension Lo. The insert 100 can include two lines of symmetry such that the right and left sides of the insert 100 on either side of the longitudinal line Lo are the same and the upper and lower portions of the insert 100 on either side of the lateral line La are the same. The insert 100 can include just one line of symmetry such that the right and left sides of the insert 100 on either side of the longitudinal line Lo are the same and the upper and lower portions of the insert 100 on either side of the lateral line La are not.

FIG. 8C illustrates a similar implementation of an insert 100 formed of a dual layer of amnion-chorion and having a non-circular inner edge 103 and outer edge 101 as in FIG. 8B. The inner diameter of the insert 100 along the longitudinal dimension Lo is about 16 mm-25 mm, preferably around 20 mm-21 mm and the inner diameter of the insert 100 along the lateral dimension La is about 14 mm-24 mm, preferably about 18 mm. The upper lobe 120 on the upper end is wider than the lower lobe 122 on the lower end along the longitudinal dimension Lo. For example, the first lobe 120 on the upper end of the insert 100 can be about 2 mm-6 mm wide, preferably about 4.5 mm wide between the inner edge 103 and the outer edge 101 whereas the second lobe 122 on the lower end of the insert 100 can be just about 1 mm-4 mm, preferably just 2 mm wide between the inner edge 103 and the outer edge 101. The width between the inner edge 103 and the outer edge 101 at the lateral sides of the insert can be even narrower at about 1 mm wide. Thus, the shape of the inner edge 103 need not resemble the shape of the outer edge 101 and the window 105 formed by the inner edge 103 need not be centrally positioned relative to the insert 100 overall. The insert 100 can include just one line of symmetry such that the right and left sides of the insert 100 on either side of the longitudinal line Lo are the same and the upper and lower portions of the insert 100 on either side of the lateral line La are not.

FIG. 8D illustrates another implementation of an insert 100 formed of a dual layer of amnion-chorion. The insert 100 of FIG. 8D has an outer edge 101 and an inner edge 103, inside of the outer edge 101. The inner edge 103 and the outer edge 101 resemble one another in overall shape, which is substantially non-circular. The insert 100 is illustrated as it would appear positioned on an eye with a maximum longitudinal dimension Lo or height greater than a maximum lateral dimension La or width. The maximum longitudinal dimension Lo can be 15-35 mm, preferably about 25 mm and the maximum lateral dimension La can be 15-25 mm, preferably about 20 mm. The inner edge 103 defines an opening or window 105 that is substantially centrally placed relative to the insert 100 overall although the window 105 may also be positioned relative to the insert 100 so that it is not substantially centered. The inner diameter of the insert 100 along the longitudinal dimension can be about 15-25 mm, preferably about 18 mm, and the inner diameter of the insert 100 along the lateral dimension La can be about 15-25 mm, preferably about 18 mm.

The inner edge 103 defining the window 105 and the outer edge 101 defining the perimeter of the insert 100 can have an obround or discorectangle shape in which the sides are substantially straight and the upper and lower ends are substantially curved. Each of the inner edge 103 and the outer edge 101 where the substantially straight sides meet the substantially curved ends have a radius of curvature. The radius of curvature for the inner edge 103 may substantially match the radius of curvature for the outer edge 101, for example, about 0.1-0.8 cm, preferably about 0.4 cm.

As with the implementation shown in FIG. 8A, the insert 100 of FIG. 8D has a first lobe 120 located on an upper end of the insert 100 along the maximum longitudinal dimension Lo and a second lobe 122 located on a lower end of the insert along the maximum longitudinal dimension Lo. The upper and lower lobes 120, 122 provide discrete regions around the circumference of the insert 100 of enlarged widths W between the inner edge 103 and the outer edge 101 that are arranged to be positioned under the lids away from the lid margins. The greater width W of the upper lobe 120 can, but need not be the same as the size of the lower lobe 122. The upper and lower lobes 120, 122 have a width W between the inner edge 103 and the outer edge 101 that is greater than the width at the sides. For example, the lobe 120 on the upper end of the insert 100 can be about 2.0 mm to about 4.5 mm wide between the inner edge 103 and the outer edge 101 whereas the width between the inner edge 103 and the outer edge 101 at the more centrally located lateral sides of the insert can be narrower at about 1 mm wide. The upper and lower lobes 120, 122 can have the same width W as one another or can have a different width W as one another such that the first lobe 120 on the upper end can be narrower or wider than the second lobe 122 at the lower end of the insert along the maximum longitudinal dimension Lo. The insert 100 can include two lines of symmetry such that the right and left sides of the insert 100 on either side of the longitudinal line Lo are the same and the upper and lower portions of the insert 100 on either side of the lateral line La are the same. The insert 100 can include just one line of symmetry such that the right and left sides of the insert 100 on either side of the longitudinal line Lo are the same and the upper and lower portions of the insert 100 on either side of the lateral line La are not.

FIG. 8E illustrates an implementation of an insert 100 formed of a dual layer of amnion-chorion in which the upper lobe 120 is wider than the lower lobe 122. The upper lobe 122 can be about 3.5 mm and the lower lobe 122 can be about 2 mm such that the window 105 is not positioned centrally relative to the overall shape of the insert 100, but rather off-set slightly toward the lower lobe 122. The shape of the window 105 in this implementation is substantially non-circular as is the shape of the outer edge 101. The insert 100 in this implementation includes one line of symmetry such that the right and left sides of the insert 100 on either side of the longitudinal line Lo are the same and the upper and lower portions of the insert 100 on either side of the lateral line La are different.

The lobes 120, 122 of the insert 100 in any of the implementations described herein, including the implementations shown in FIGS. 7A-7D or FIGS. 8A-8G, that are sized to be placed subpalpebrally can additionally incorporate one or more holes, slots, cut-outs, pores, or other surface features 124. The features 124 allow for the lobes 120, 122 to be used by the operator to position the insert 100 on the anterior surface of the eye, such as with a cotton swab or Weck-Cel eye spear used to position the insert 100 onto the eye. The tool(s) used to position the insert 100 can manipulate the insert 100 by interfacing with these surface features 124. Additionally, the surface features 124 can be configured to allow for fluid egress through the insert 100 upon positioning on the eye. The surface features 124 prevent fluid from becoming trapped between the lower surface of the insert 100 and the surface of the eye thereby improving the contact between the insert 100 and the ocular surface and reducing the risk of slippage.

FIG. 8F illustrates an implementation of an insert 100 formed of a dual layer of amnion-chorion. The insert 100 has an outer edge 101 and an inner edge 103 inside of the outer edge 101. The inner edge 103 defines an opening or window 105, which can be centrally positioned relative to the insert 100 or non-centrally positioned relative to the insert 100. The outer edge 101 defines a substantially non-circular shape to the insert 100. The inner edge 103 defining the window 105 is also substantially non-circular in shape such that it has a greater height than its width. The window 105 may also be substantially circular in shape. As with other implementations, the insert 100 has an upper lobe 120 located on an upper end of the insert 100 along the maximum longitudinal dimension Lo and a lower lobe 122 located on a lower end of the insert along the maximum longitudinal dimension Lo. The upper and lower lobes 120, 122 provide discrete regions around the circumference of the insert 100 with enlarged widths W between the inner edge 103 and the outer edge 101 that are arranged to be positioned under the lids away from the lid margins. The upper and lower lobes 120, 122 can have the same width W as one another or can have a different widths as one another such that the first lobe 120 on the upper end can be narrower or wider than the second lobe 122 at the lower end of the insert along the maximum longitudinal dimension Lo. Each lobe 120, 122, however, is greater than the width at the more centrally located lateral sides. For example, the lobes 120, 122 can be about 3.5 mm wide (e.g., about 2 mm to about 5 mm) whereas the sides can be about 1 mm (e.g., about 0.5 mm to about 2.5 mm). One or both of the upper and lower lobes 120, 122 can incorporate at least one surface feature 124 that is in the shape of an elongate cut-out. The cut-out surface feature 124 can extend from the outer edge 101 toward the inner edge 103. Where the lobe is about 3.5 mm wide, the cut-out can have a width that is about 0.5 mm and have a depth that is at least about 1 mm away from the inner edge 101 so as not to extend clear to the inner edge 101.

The insert 100 of FIG. 8F can include two lines of symmetry such that the right and left sides of the insert 100 on either side of the longitudinal line Lo are the same and the upper and lower portions of the insert 100 on either side of the lateral line La are the same. The implementation shown in FIG. 8F shows the upper lobe 122 includes a first surface feature 124 that is a cut-out and a second surface feature 124 that is a cut-out positioned a distance away from the first surface feature 124 on an opposite side of longitudinal line Lo. Each surface feature 124 can be positioned about 60 degrees from the lateral line La. The lower lobe 122 is shown having the same arrangement of surface features 124 as the upper lobe 120 although it should be appreciated that the upper lobe 120 can include greater or fewer surface features 124 as the lower lobe 122. For example, FIG. 8A shows that each lobe 120, 122 has just one surface feature 124, which is in the shape of a cut-out or hole.

FIG. 8G illustrates an interrelated implementation of an insert 100 formed of a dual layer of amnion-chorion and having a plurality of surface features 124. Each lobe 120, 122 can include a plurality of surface features 124 arranged at an angle relative to one another. For example, the upper lobe 120 can have a first surface feature 124 arranged about 0 degrees relative to or coaxial with a longitudinal line Lo, a second surface feature 124 can be arranged at about 11 degrees relative to the longitudinal line Lo, a third surface feature 124 arranged at about 22 degrees relative to the longitudinal line Lo, a fourth surface feature 124 arranged at about 33 degrees relative to the longitudinal line Lo, and a fifth surface feature 124 arranged at about 44 degrees relative to the longitudinal line Lo, and a sixth surface feature 124 arranged at about 55 degrees relative to the longitudinal line Lo. The opposite side of the upper lobe 120 can have a similar arrangement of surface features 124. A seventh surface feature 124 can be arranged at about 11 degrees relative to the longitudinal line Lo in an opposite direction, an eight surface feature 124 arranged at about 22 degrees relative to the longitudinal line Lo in an opposite direction, a ninth surface feature 124 arranged at about 33 degrees relative to the longitudinal line Lo in an opposite direction, and a tenth surface feature 124 arranged at about 44 degrees relative to the longitudinal line Lo in an opposite direction, and an eleventh surface feature 124 arranged at about 55 degrees relative to the longitudinal line Lo in an opposite direction. Thus, the upper lobe can have up to about eleven surface features arranged symmetrically around the longitudinal line Lo. The lower lobe can have the same arrangement of surface features as illustrated in FIG. 8G.

The surface features 124 can avoid penetrating both the inside edge 103 and the outside edge 101 such that the surface features 124 form holes through the lobes 120, 122 (see, e.g., FIGS. 8A and 8G). The size of the holes can vary as can the shape. In some implementations, the holes are rectangular or substantially elongate in shape such that they are longer than they are wide. In an implementation, the holes are about 0.75 mm wide and about 3 mm long (see FIG. 8A). In an implementation, the holes are about 0.5 mm wide and about 2.2 mm long (see FIG. 8G). The holes can change in overall length moving toward the narrower sides of the insert 100 such that the holes arranged closer to the longitudinal line Lo have a greater length (e.g., 2.0-2.2 mm) compared to the holes arranged further away from the longitudinal line Lo (e.g., 1.4-1.7 mm). This ensures a minimal thickness is left within the lobe on either end of the holes (e.g., about 1-1.5 mm).

Upon positioning the insert 100 onto the eye, the bio-tissue of the insert 100 can absorb fluids of the eye and rehydrate. Such fluids can be topically delivered medications (e.g., eye drops) which will be absorbed and retained into the amniotic biotissue which can then serve as a sustained release depot to increase the bio-availability of the medications to the ocular surface.

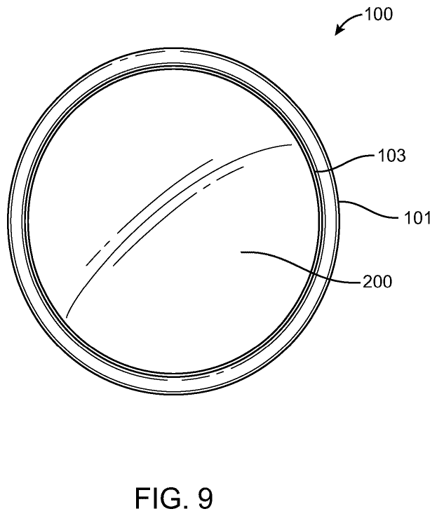

In some implementations, the insert 100 can incorporate a separate retention feature 200 configured to provide structural stability to the insert. The configuration of the retention feature 200 can vary, including a separate anchor or other retaining structure, a bandage, one or more punctal plugs, or standard corneal contact lens. FIG. 9 shows an insert 100 coupled to a retention feature 200 that is a corneal contact lens having an outer perimeter sized to be received within the opening 105 of the insert 100 so that the insert 100 conforms to the outer perimeter of the corneal contact lens. The inner edge 103 of the insert 100 is greater than a diameter of the outer perimeter of the retention feature 200. Thus, the peripheral edge of the corneal contact lens serves as a retaining element to the insert 100 to keep the insert 100 centered on the anterior surface of the eye. The amniotic membrane insert 100 can be the same thickness or thinner than the retention feature 200. The insert 100 thus encircles but does not snag the palpebral surface. The window 105 of the insert 100 can have a diameter that is 0.1-2 mm greater than the diameter of the outer perimeter of the retention feature 200. The insert 100 can have a thickness that is less than 4 mm and/or thinner than a thickness of the retention feature 200. The retention feature 200 can remain in place on the eye as long as the insert 100 remains in place. The retention feature 200 can biodegrade before or after the insert 100 is no longer positioned on the eye. Additionally, the insert inner edge can have a larger diameter than the cornea and the retention feature can be completely transparent resulting in no obstruction of vision while still allowing the amniotic membrane to release the bioactive factors into the tear film.

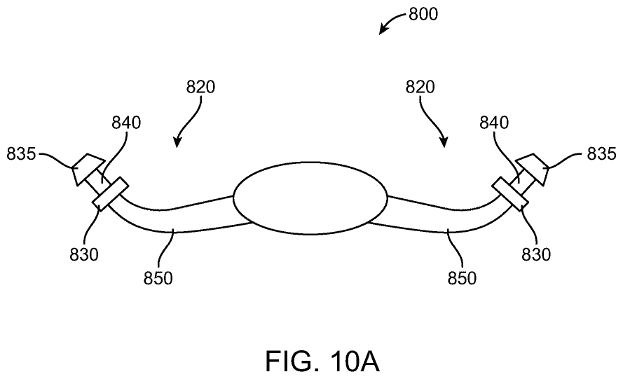

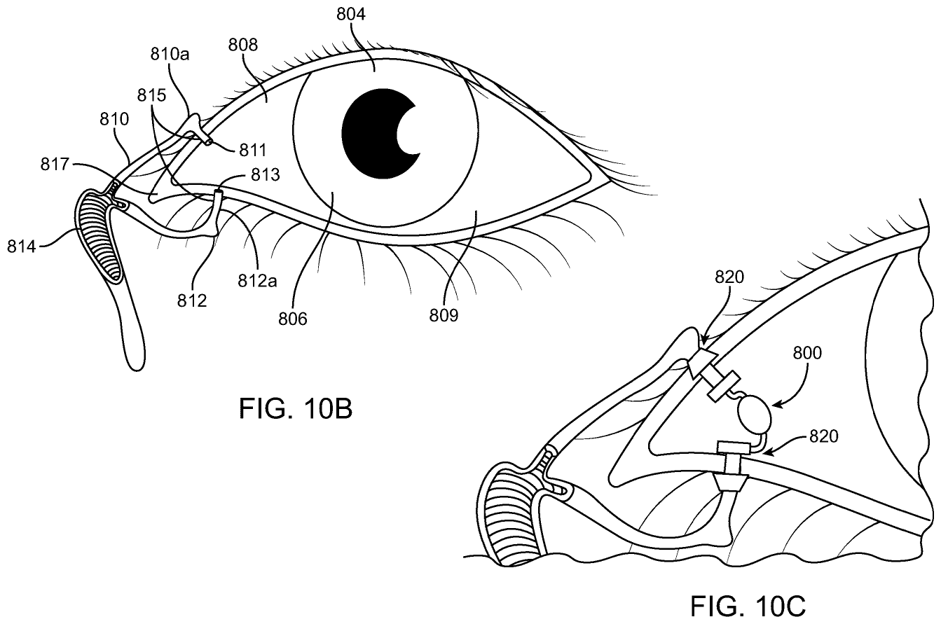

The biotissue insert having a separate retention feature need not be annular in shape nor encircle the globe of the eye and the retention feature need not cover or contact the cornea. FIG. 10A shows a biotissue insert 800 coupled to at least one retention feature 820. The insert 800 can be a segment of amnion, chorion, amnio-chorion as described elsewhere herein. The insert 800 can have any of a variety of shapes and need not be annular, including a strip, disc, or other segment of tissue sized to contact at least a portion of the ocular surface. The retention feature 820 can be in the form of a punctal plug coupled to the insert 800 by a tissue connector 850. The biotissue insert 800 can be positioned using at least one retention feature 820. FIGS. 10A and 10C show a biotissue insert 800 positioned using a pair of retention features 820, each of the retention features 820 being in the shape of a punctal plug and arranged to insert in each of the upper and lower punctum.

FIG. 10B shows an eye including a cornea 804, an iris 806, and a sclera 808 surrounding the cornea 804 and iris 806. A conjunctival layer 809 is substantially transparent and disposed over sclera 808. The lacrimal system includes an upper canaliculus 810 and a lower canaliculus 812, collectively the canaliculae, and the nasolacrimal duct or sac 814. The upper and lower canaliculae 810, 812 terminate in an upper punctum 811 and a lower punctum 813, respectively, which are also referred to as punctal apertures. The punctal apertures 811, 813 are situated on a slight elevation at the medial end of the lid margin at the junction 815 of the ciliary and lacrimal portions near the medial canthus 817. The punctal apertures 811, 813 are round or slightly ovoid openings surrounded by a connective ring of tissue. Each of the punctal openings 811, 813 leads into a vertical portion 810a, 812a of the respective canaliculus before turning horizontally to join its other canaliculus at the entrance of a lacrimal sac 814. The canaliculae 810, 812 are tubular and lined by stratified squamous epithelium surrounded by elastic tissue which permits the canaliculus to be dilated.

The punctal plug retention feature 820 is shaped to insert at least in part within one of the punctal apertures 811, 813 so that the insert 800 is positioned on a surface of the eye near the puncta (see FIG. 10C). The retention feature 820 can include a first punctal plug coupled to the insert 800 by a first tissue connector 850, the first punctal plug being positioned within a first puncta, and a second punctal plug coupled to the insert 800 by a second tissue connector 850, the second punctal plug being positioned within a second puncta. The biotissue insert 800 can be coupled to each of the first and second punctal plugs so that the insert 800 spans generally between the retention features 820.

The retention feature 820 having a shape of a punctal plug can incorporate a collar 830 at a proximal end, a tapered portion 835 near a distal end, and a body portion 840 extending between the collar 830 and the tapered portion 835. The tapered portion 835 is sized and shaped to project into the canaliculus 810, 812. The taper angle of the tapered portion 835 eases insertion of the tapered portion into the punctum 811, 813. The tapered portion 835 can have a proximal ledge feature 840 that is designed to prevent the plug from being inadvertently dislodged (i.e., withdrawn) from the canaliculus. The collar 830, in use, is sized and shaped to rest against the exterior of a punctum 811, 813. The collar 830 has a diameter sized to prevent the collar 830 from entering the canaliculus. It is also designed to minimize irritation to the eye. The body portion 840 extending between the collar 830 and the tapered portion 835 is sized and shaped to reside comfortably within the canaliculus 810, 812. The collar 830, tapered portion 835, and body portion 840 can each be formed from a flexible material, such as silicone, that is impermeable to fluids. The material is biocompatible and sufficiently flexible to mold into shape within the canaliculus. The tissue connector 850 can project proximally from the collar 830 (i.e., away from the distal end of the tapered portion 835 located within the canaliculus). The tissue connector 850 couples the biotissue insert 800 to a region of the collar 830 or other portion of the plug that remains outside the punctum so that the insert 800 is fixed relative to a surface of the eye up insertion of the body portion 840 into the canaliculus 810, 812. The tissue connector 850 can be formed of any of a variety of materials, including suture, molded silicone, or other biocompatible material. The tissue connector 850 can be connected to the plug with adhesive or by a mechanical clamp or by molding onto the tissue, with a feature in the amniotic membrane such as a hole or holes that mate with features on the tissue connector). At least one punctal plug or each plug as well as the tissue connector can be constructed of amniotic membrane. In one implementation, the entire plug, tissue connector, and insert construct is composed of a single continuous sheet of amniotic membrane that is cut and processed into the shape. The retention feature 820 having a shape of a punctal plug (e.g., a collar 830 at a proximal end, a tapered portion 835 near a distal end, and a body portion 840 extending between the collar 830 and the tapered portion 835) can be formed entirely of biotissue, such that the retention feature 820 (optionally including the tissue connector 850) and the insert 800 are each formed entirely of biotissue.

The body portion 840 can, but need not, be tubular and extend between two apertures allowing fluid flow through the plug. In preferred implementations, the body portion 840 is solid and the plug includes no apertures so that no fluid flows through the retention feature 820 so that the plug prevents tear film drainage. Punctal occlusion prevents drainage of the tear film through the lacrimal system thereby enhancing tear film preservation, which is particularly useful for treating dry eye. The increased tear film also improves bioelution from the biotissue insert 800, which is positioned in contact with the medial ocular surface around or on top of the caruncle. The biotissue insert 800 can be positioned under the lid within the cul-de-sac of the eye and retained by a single punctal plug retention feature 820. Bi-canalicular cross-punctal fixation of a biotissue insert provides sutureless, self-retaining anchoring of the device on the ocular surface and provides the benefit of punctal occlusion and ocular surface bio-elution.

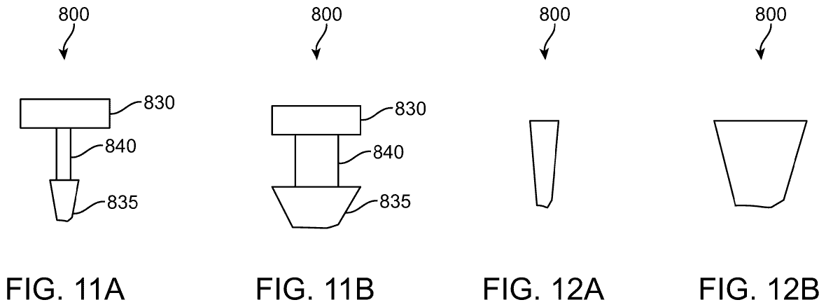

In some implementations, the insert 800 has the shape of a punctal plug (see FIGS. 11A-11B). The punctal plug shape can incorporate a collar 830 at a proximal end, a tapered portion 835 near a distal end, and a body portion 840 extending between the collar 830 and the tapered portion 835. At least the body portion 840 that is designed to be positioned within the punctum is formed of biotissue (e.g., scleral tissue, corneal tissue, amniotic membrane tissues having one or more layers, amnion-chorion membranes having one or more layers, etc.). The tapered portion 835 near the distal end can additionally be formed of biotissue. The collar 830 can be formed of biotissue as well such that the entire insert 800 is biotissue. Alternatively, the collar 830 can be another material that is not biotissue.

Biotissue that is dehydrated can have a first dimension that increases upon rehydration due to swelling. The dehydrated biotissue can serve as an occlusive punctal plug upon implantation and rehydration within the punctum. The insert 800 formed of biotissue can be inserted within the punctum in a first state in which the biotissue is dehydrated and has a first dimension (see FIG. 11A). After implantation into the punctum and rehydration of the biotissue, the insert 800 can transition to a second, larger dimension that wedges into and occludes the punctum (see FIG. 11B).

The shape of the biotissue insert 800 for implantation within the punctum need not have the shape shown with the tapered portion, body portion, and collar. For example, the biotissue insert 800 can be shaped into a triangle or wedge shape having the narrower part arranged more distally for ease of insertion into the punctum and the wider part arranged more proximally for better occlusion of the punctum (see FIG. 12A-12B). The shape of the biotissue insert can also be largely rectangular or cylindrical, with a consistent cross-sectional area. The biotissue insert can also consist of two or more separate biotissue bodies, wherein each body can be completely composed of biotissue, or one or more bodies can be composed of biotissue combined with other synthetic materials.

As described elsewhere herein, the biotissue insert 800 for occluding the punctum can include one or more layers of biotissue. The bio-tissue insert 800 can include multi-layer constructs including amnion-chorion, amnion-amnion, chorion-chorion, amnion-chorion-amnion, amnion-chorion-chorion-amnion, chorion-amnion-amnion-chorion, amnion-amnion-amnion (triple layer), and any of a variety of combinations of multilayer constructs of the amnion and chorion. Preferably, the biotissue insert 800 is a multilayer amnion-chorion-chorion-amnion sandwich membrane.

The biotissue insert 800 can increase in one or more dimensions by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, up to about 250% and any number in between. In the first dehydrated state, the biotissue insert 800 has a thickness of about 25 microns to 1400 microns. In the second, rehydrated state, the biotissue insert 800 thickness increases to about 50 microns to 1000 microns. The biotissue insert 800 can increase additionally or alternatively in width from 25 microns to 700 microns wide, preferably 75-350 microns wide, in the first dehydrated state to about 50 microns to 1400 microns, preferably about 150-700 microns wide, in the second rehydrated state. The dimension of the bio-tissue can vary along its length, with a narrower portion inserted first into the punctum, and one or more wider portions along the length.

The biotissue insert 800 can be formed by cutting or punching out tissue into a desired shape (or shapes where a kit of multiple inserts is considered). The biotissue insert 800 can be formed by folding biotissue into a desired shape. The biotissue insert 800 can be formed by rolling biotissue into a desired shape. The biotissue insert 800 can be formed by laser cutting biotissue into a desired shape(s).

Upon insertion within the punctum, at least a portion of the biotissue insert 800 can be located inside the punctum and at least a portion of the biotissue insert 800 can be located outside the punctum, such as for ease of handling and insertion. The portion of the insert 800 remaining outside the punctum can be trimmed following insertion of the insert 800 within the eye to avoid excessive material remaining during use. In other implementations, the portion of the insert 800 remaining outside the punctum can be shaped into a flap or pad or collar that can be positioned within the caruncle just outside the punctum opening. This proximal flap, pad, or collar can be sized to extend beyond the caruncle onto the ocular surface.

### EXPERIMENTAL

Initial studies were conducted to assess tolerance of the amniotic tissue insert in patients. The insert made of amniotic tissue cut into a ring shape having a 24-mm inner diameter and 27-mm outer diameter. Other insert sizes evaluated had 21-mm inner diameter and 25-mm outer diameter. The insert was positioned on the anterior surface of the eye, with or without topical anesthesia, using fingers and a cotton swab to adjust positioning. The inserts were well tolerated by patients, even those without prior experience using contact lenses or glasses. Patients were unable to sense the presence of the insert by about 5 minutes post-placement. In some instances where the insert folded slightly at the lateral canthus, patients experienced sensation of the insert that resolved upon flattening the lower surface of the insert against the eye. The insert was nearly invisible on the anterior surface of the eye due to it blending into the conjunctiva. The inserts were easily removed after 30 minutes with fingers. The inserts were retained without any inadvertent slippage or folding.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal.” Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference and are not intended to limit the use or orientation of the devices to a specific configuration described in the various implementations.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value. In aspects, about includes the specified value. One inch or 1″ corresponds to 2.54 cm (SI-units).

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The systems disclosed herein may be packaged together in a single package. The finished package would be sterilized using sterilization methods such as Ethylene oxide or radiation and labeled and boxed. Instructions for use may also be provided in-box or through an internet link printed on the label.

1.-54. (canceled)

55. A punctal plug formed of a biotissue having a size and a shape to insert, at least in part, within a punctum of an eye.

56. The punctal plug of claim 55, wherein the biotissue occludes the punctum.

57. The punctal plug of claim 55, wherein the biotissue increases a tear film of the eye upon being inserted within the punctum.

58. The punctal plug of claim 55, wherein the biotissue is scleral tissue or corneal tissue.

59. The punctal plug of claim 55, wherein the biotissue is amniotic membrane tissue.

60. The punctal plug of claim 59, wherein the amniotic membrane tissue is a multi-layer construct that includes an amnion layer and a chorion layer.

61. The punctal plug of claim 60, wherein the multi-layer construct of amnion and chorion is a dual layer, triple layer, or quadruple layer.

62. The punctal plug of claim 60, wherein the multi-layer construct comprises a dual layer of amnion-chorion, chorion-amnion, amnion-amnion, or chorion-chorion.

63. The punctal plug of claim 60, wherein the multi-layer construct comprises a triple layer of amnion-amnion-amnion, chorion-amnion-amnion, amnion-chorion-amnion, amnion-amnion-chorion, chorion-chorion-amnion, amnion-chorion-chorion, chorion-amnion-chorion, or chorion-chorion-chorion.

64. The punctal plug of claim 60, wherein the multi-layer construct comprises a quadruple layer of amnion-chorion-chorion-amnion or chorion-amnion-amnion-chorion.

65. The punctal plug of claim 59, wherein the biotissue bioelutes one or more healing factors into a tear film of the eye.

66.-69. (canceled)

70. The punctal plug of claim 55, wherein the biotissue is dehydrated in a first state having the size.

71. The punctal plug of claim 70, wherein the biotissue rehydrates upon deployment in the punctum and transitions towards a second state that is a larger in at least one dimension.

72. The punctal plug of claim 71, wherein the biotissue wedges into and occludes the punctum upon transitioning towards the second state.

73. The punctal plug of claim 55, wherein the shape of the biotissue is triangular with a narrower dimension toward a distal end and a wider dimension toward a proximal end.

74. The punctal plug of claim 55, wherein the shape of the biotissue is rectangular or cylindrical.

75. The punctal plug of claim 55, wherein the punctal plug is formed of two or more separate bodies of the biotissue.

76. The punctal plug of claim 55, wherein the biotissue increases in one or more dimensions upon deployment in the punctum by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, up to about 250%.

77.-82. (canceled)

83. The punctal plug of claim 55, wherein the biotissue is cut, rolled, and/or folded into the size and the shape.

84.-86. (canceled)
